(19) United States
(12) Patent Application Publication
(10) Pub. No.: US 2026/0130974 A1
(43) Pub. Date: May 14, 2026

(54) FACTOR IX VARIANT POLYPEPTIDES FOR ADMINISTRATION TO SOFT TISSUE

(71) Applicant: CSL Innovation Pty Ltd, Melbourne, Victoria (AU)

(72) Inventors: Steffi Knoll MACHADO, Marburg (DE); Padmapriya PONNUSWAMY, Marburg (DE); Sabine PESTEL, Marburg (DE); Philipp CLAAR, Marburg (DE)

(21) Appl. No.: 19/119,611

(22) PCT Filed: Oct. 6, 2023
(86) PCT No.: PCT/AU2023/050980

§ 371 Date: Apr. 9, 2025

(30) Foreign Application Priority Data
Oct. 10, 2022 (EP) 22200505.0

(51) Int. Cl. A61K 38/48; A61P 7/04

(52) U.S. Cl. CPC A61K 38/4846; A61P 7/04; C12Y 304/21022

(57) ABSTRACT

This invention relates to Factor IX variant polypeptides for administration to soft tissues.

### RELATED APPLICATION DATA

The present application claims priority from European Patent Application No. EP22200505.0 entitled “Factor IX variant polypeptides for administration to soft tissue” filed on 10 Oct. 2022, the entire contents of which is hereby incorporated by reference.

### SEQUENCE LISTING

The present application is filed with a Sequence Listing in electronic form. The entire contents of the Sequence Listing are hereby incorporated by reference.

### TECHNICAL FIELD

This invention relates to Factor IX (FIX) variant polypeptides for administration to soft tissues, such as skin tissue (including subcutaneous administration) or mucosal tissue (e.g., gastrointestinal mucosal tissue), and their use in therapy. In particular, described herein are FIX variant polypeptides that have increased hemostatic efficacy when administered to soft tissues, compared to wild-type FIX.

### BACKGROUND

Human coagulation FIX plays a key role in the formation of blood clots. FIX has been used in the prophylaxis and treatment of bleeding disorders, such as hemophilia B. Maintenance of an appropriate level of FIX activity in the plasma is crucial for preventing bleeding in hemophilia B patients. If left untreated, lack of FIX activity can cause severe damage to the patient and can even lead to death.

The prevailing view in the field is that increasing the circulation time of the FIX proteins in plasma is important for maintaining an appropriate level of FIX activity and to achieve hemostasis. Hemostasis is the mechanism that leads to cessation of bleeding from a blood vessel. Current treatments for hemophilia B therefore include the intravenous administration of FIX proteins that have an extended half-life in plasma, including IDELVION®, ALPROLIX® and REBYNIN®.

Gene therapy is currently being investigated for the treatment of hemophilia B. Current approaches use adeno-associated virus (AAV) vectors to deliver the FIX transgene (ref 1). This approach has already been utilized in clinical trials with a significant reduction of annualized bleeding rates over the course of 5 years following treatment with adeno-associated virus expressing a highly active variant of FIX (ref 2). However, certain patients may be more suited to FIX protein replacement therapy rather than gene therapy.

Recently, the relevance of the extravascular FIX reservoir has emerged as a concept that may contribute to hemostasis. The extravascular FIX reservoir refers to non-circulating FIX that is bound outside the plasma, in the extravascular space, such as the vascular endothelium or subendothelial extracellular matrix. Understanding the balance between extravascular FIX and circulating FIX opens new possibilities to identify novel and improved strategies for treating hemophilia B. The present invention is based on the surprising realisation that the hemostatic efficacy of FIX when administered specifically to soft tissue (including subcutaneous administration) can be improved by using certain FIX variant polypeptides that have reduced binding to extracellular matrix.

### DISCLOSURE OF THE INVENTION

The present invention provides advantageous FIX variant polypeptides which have increased hemostatic efficacy when administered specifically to a soft tissue, for example when administered subcutaneously. The invention is particularly suitable for use with FIX variant polypeptides that have decreased binding to extracellular matrix, such as the K5A variant, compared to wild-type FIX. The K5A variant, for example, had been described previously (ref 3), but the surprising effect of improved hemostatic efficacy when administered specifically to soft tissue had not been demonstrated. As now shown in the present disclosure, the inventors have advantageously identified that when these FIX variant polypeptides are administered to a soft tissue, they enter circulation more easily. The examples demonstrate that this results in a higher level of bioavailable FIX in the plasma, which results in significantly improved hemostatic efficacy, as compared to administering a wild-type FIX by the same route, or administering the same variant but via the intravenous route, a finding that is particularly surprising. Therefore, these FIX variant polypeptides are particularly useful for treating and preventing bleeding disorders, such as hemophilia B, when administered to a soft tissue.

In one aspect, the invention therefore provides a Factor IX (FIX) variant polypeptide for use in a method of treating or preventing a bleeding disorder comprising administering the FIX variant polypeptide to a soft tissue, wherein the FIX variant polypeptide comprises the amino acid alanine at a position corresponding to position 5 of wild-type Factor IX. SEQ ID NO: 1 is an example of a wild-type FIX polypeptide sequence as referred to herein and below.

As an alternative to any of the aspects and embodiments described herein that use a FIX variant polypeptide comprising the amino acid alanine at a position corresponding to position 5 of wild-type Factor IX (‘K5A’), the FIX variant polypeptide may instead comprise the amino acid lysine at a position corresponding to position 10 of wild-type Factor IX (‘V10K’). Both variants have been shown to have decreased binding to extracellular matrix, as compared to wild-type FIX (ref 4).

The invention also provides a method of treating or preventing a bleeding disorder in a subject comprising administering a therapeutically or prophylactically effective amount of a FIX variant polypeptide to a soft tissue in the subject, wherein the FIX variant polypeptide comprises the amino acid alanine at a position corresponding to position 5 of wild-type Factor IX.

The invention further provides a use of a FIX variant polypeptide in the manufacture of a medicament for treating or preventing a bleeding disorder in a subject, wherein the FIX variant polypeptide is to be administered to a soft tissue in the subject and wherein the FIX variant polypeptide comprises the amino acid alanine at a position corresponding to position 5 of wild-type Factor IX.

The invention further provides a FIX variant polypeptide for treating or preventing a bleeding disorder, wherein the FIX variant polypeptide is to be administered to a soft tissue in the subject and wherein the FIX variant polypeptide comprises the amino acid alanine at a position corresponding to position 5 of wild-type Factor IX.

In some embodiments, the bleeding disorder is hemophilia B (also known as congenital factor IX deficiency).

In some embodiments, the FIX variant polypeptide further comprises (i.e., in addition to the amino acid alanine at a position corresponding to position 5 of wild-type Factor IX) the amino acid lysine at a position corresponding to position 10 of wild-type Factor IX.

In some embodiments, the FIX variant polypeptide further comprises (i.e., in addition to the amino acid alanine at a position corresponding to position 5 of wild-type Factor IX, and optionally the amino acid lysine at a position corresponding to position 10 of wild-type Factor IX), the amino acid leucine at a position corresponding to position 338 of wild-type Factor IX.

In other embodiments, the FIX variant polypeptide further comprises (i.e., in addition to the amino acid alanine at a position corresponding to position 5 of wild-type Factor IX, and optionally the amino acid lysine at a position corresponding to position 10 of wild-type Factor IX), an amino acid other than arginine (e.g., an amino acid selected from the group consisting of valine, threonine and tryptophan) at a position corresponding to position 338 of wild-type Factor IX in combination with the amino acid histidine at a position corresponding to position 410 of wild-type Factor IX. In certain such embodiments, the FIX variant polypeptide comprises valine at a position corresponding to position 338 of wild-type Factor IX and the amino acid histidine at a position corresponding to position 410 of wild-type Factor IX.

In some embodiments, the FIX variant polypeptide further comprises (i.e., in addition to the amino acid alanine at a position corresponding to position 5 of wild-type Factor IX, and optionally the amino acid lysine at a position corresponding to position 10 of wild-type Factor IX) the amino acid tyrosine at a position corresponding to position 318 of wild-type Factor IX, the amino acid glutamic acid at a position corresponding to position 338 of wild-type Factor IX and the amino acid arginine at a position corresponding to position 343 of wild-type Factor IX. The Factor IX variant polypeptide can have an amino acid sequence that is at least 70%, 80%, 90%, 95%, 96%, 97%, 98% or at least 99% identical to SEQ ID NO: 1, across the full length of SEQ ID NO: 1.

In any of the embodiments described herein, the Factor IX variant polypeptide can have the sequence of SEQ ID NO: 1 except for the substitutions that are specified herein (e.g., SEQ ID NO: 1 with lysine at position 5 substituted with alanine, etc.).

In some embodiments, the FIX variant polypeptide comprises a half-life enhancing portion, such as albumin including variants and derivatives thereof, polypeptides of the albumin family including variants and derivatives thereof, immunoglobulins without antigen binding domain (e.g., the Fc portion only) or polyethylene glycol.

In some embodiments, the FIX variant polypeptide further comprises a cleavable peptide linker between the FIX variant polypeptide and the half-life enhancing portion.

In some embodiments, the soft tissue is skin tissue or gastrointestinal tract tissue (e.g., mucosal gastrointestinal tract tissue). In certain embodiments, the soft tissue is skin tissue, including the subcutaneous tissue. In certain such embodiments, the FIX variant polypeptide is subcutaneously administered. For example, the FIX variant polypeptide is for use in a method of treating or preventing a bleeding disorder comprising administering the FIX variant polypeptide subcutaneously, wherein the FIX variant polypeptide comprises the amino acid alanine at a position corresponding to position 5 of wild-type Factor IX.

In alternative embodiments, the FIX variant polypeptide is administered into the gastrointestinal tract tissue using an oral drug delivery device. For example, the FIX variant polypeptide is for use in a method of treating or preventing a bleeding disorder comprising administering the FIX variant polypeptide into the gastrointestinal tract tissue using an oral drug delivery device, wherein the FIX variant polypeptide comprises the amino acid alanine at a position corresponding to position 5 of wild-type Factor IX.

Another aspect of the invention provides a pharmaceutical composition comprising a FIX variant polypeptide for use in a method of treating or preventing a bleeding disorder comprising administering the pharmaceutical composition to a soft tissue, wherein the FIX variant polypeptide comprises the amino acid alanine at a position corresponding to position 5 of wild-type Factor IX.

The invention also provides a method of treating or preventing a bleeding disorder in a subject comprising administering a therapeutically or prophylactically effective amount of a pharmaceutical composition comprising a FIX variant polypeptide to a soft tissue in the subject, wherein the FIX variant polypeptide comprises the amino acid alanine at a position corresponding to position 5 of wild-type Factor IX.

The invention further provides a use of a pharmaceutical composition comprising a FIX variant polypeptide in the manufacture of a medicament for treating or preventing a bleeding disorder in a subject, wherein the pharmaceutical composition is to be administered to a soft tissue in the subject and wherein the FIX variant polypeptide comprises the amino acid alanine at a position corresponding to position 5 of wild-type Factor IX.

The invention further provides a pharmaceutical composition comprising a FIX variant polypeptide for treating or preventing a bleeding disorder, wherein the pharmaceutical composition is to be administered to a soft tissue in the subject and wherein the FIX variant polypeptide comprises the amino acid alanine at a position corresponding to position 5 of wild-type Factor IX.

It will be clear that FIX variant polypeptides comprising additional mutations (i.e., in addition to the amino acid alanine at a position corresponding to position 5 of wild-type Factor IX), as disclosed herein, can be used in any of the above aspects and embodiments.

### Definitions

The practice of the present invention will employ, unless otherwise indicated, conventional methods of chemistry, biochemistry, molecular biology, immunology and pharmacology, within the skill of the art. Such techniques are explained fully in the literature.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides of this invention may be based, for example, upon antibodies or other members of the immunoglobulin superfamily, in certain embodiments, a “polypeptide” can occur as a single chain or as two or more associated chains.

A percentage sequence identity between two amino acid sequences means that, when aligned, that percentage of amino acids is the same in comparing the two sequences. The percentage sequence identity is calculated as the percentage of identical amino acids within the aligned sequences. A sequence that “has” (or “having”) x % sequence identity to another sequence means that the sequence is x % identical to that other sequence.

The term “wild-type Factor IX” refers to a Factor IX polypeptide sequence that occurs naturally and has a FIX activity that is typical of natural FIX such as that found in standard human plasma. The sequence has not been artificially modified relative to the sequence of the naturally occurring polypeptide sequence. This means that none of the amino acids in the naturally occurring polypeptide sequence has been substituted with a different amino acid. SEQ ID NO: 1 is an example of a wild-type polypeptide sequence, but functional fragments, truncations, etc. are also encompassed by the term, as exemplified below. For example, the term includes polypeptides with a modified N-terminal or C-terminal end including terminal amino acid deletions or additions, as long as those polypeptides substantially retain the activity of wild-type Factor IX. The term also includes any natural polymorphic variant of Factor IX. For example, a common natural polymorphic variant which occurs with a frequency of 33% is a Factor IX polypeptide presenting an alanine (A) in a position corresponding to position T148 in SEQ ID NO: 1. This T148A polymorphic variant is shown in SEQ ID NO: 20. All references to SEQ ID NO: 1 herein may therefore also refer to SEQ ID NO: 20. Although these polymorphic variants occur naturally in the general population at least some of them have been associated with phenotypic effects, for example the T148A has been described in the literature (ref. 56).

The terms “FIX variant polypeptide”, “FIX variant”, “variant”, “FIX polypeptide” and so on are used interchangeably herein and all refer to a FIX variant polypeptide unless expressly stated otherwise. FIX variant polypeptides include full length FIX proteins or fragments of FIX proteins that are biologically active, i.e., the polypeptide is capable of activating Factor X (i.e., generating Factor Xa). The Factor IX variant polypeptides of the invention are derived from a polypeptide sequence of wild-type Factor IX (SEQ ID NO: 1). Variants differ at one or more amino acid positions from the corresponding positions in the wild-type Factor IX, i.e., the variant has one or more amino acid substitutions relative to the corresponding positions in the wild-type Factor IX. The numbering refers to the amino acid positions in wild-type Factor IX as defined in SEQ ID NO: 1. An exemplary polynucleotide coding sequence for the polypeptide of SEQ ID NO: 1 is provided by SEQ ID NO: 2.

For the avoidance of any doubt, all of the FIX variant polypeptides described herein have FIX clotting activity, e.g., they have the clotting activity of wild-type FIX, or they may even have a higher clotting activity than wild-type FIX; clotting activity can be measured by standard assays known to those skilled in the art.

The Factor IX variant polypeptide may also be derived from a wild-type Factor IX that includes the signal and/or the propeptide, as shown in SEQ ID NO: 3. SEQ ID NO: 3 includes both the signal peptide (aa 1-28) and the propeptide (aa 29-46). The polypeptide of SEQ ID NO: 3 is known in the art as the precursor of human Factor IX, or as the prepropeptide Factor IX. Factor IX with propeptide but lacking the signal peptide is also known as a propeptide Factor IX. An exemplary polynucleotide coding sequence encoding the polypeptide of SEQ ID NO: 3 is shown in SEQ ID NO: 4.

The Factor IX variant polypeptide may also be derived from one or more functional fragments of wild-type Factor IX, for example it may be derived from activated Factor IX which contains two fragments of Factor IX (it is missing the intervening ‘activation peptide’ that is present in SEQ ID NO: 1). SEQ ID NOs 17 and 18 show the light chain and heavy chain, respectively, of human activated Factor IX, which are held together by a disulphide bridge. Another example is isoform 2 of human Factor IX, which lacks the 38-aa stretch at positions 47-84 of SEQ ID NO: 1.

Alternatively, the Factor IX variant polypeptide may be derived from a truncation or a fusion of wild-type Factor IX.

The term “derived from a polypeptide sequence of wild-type Factor IX” (or similar wording) means that the Factor IX variant polypeptide has some degree of sequence identity with wild-type Factor IX polypeptide when the two sequences are aligned. For example, the Factor IX variant polypeptide may have at least 70% etc. sequence identity to SEQ ID NO: 1, as described above. The Factor IX variant polypeptide is biologically active, i.e., it is capable of activating Factor X (i.e., generating Factor Xa).

The Factor IX variant polypeptide may be provided as an “isolated” or as a “purified” polypeptide. This term may refer to a polypeptide produced by expression of an isolated nucleic acid molecule of the invention. Alternatively, this term may refer to a protein which has been sufficiently separated from other proteins with which it would naturally be associated (e.g., so as to exist in “substantially pure” form). “Isolated” is not meant to exclude artificial or synthetic mixtures with other compounds or materials, or the presence of impurities that do not interfere with the fundamental activity, and that may be present, for example, due to incomplete purification, or the addition of stabilizers.

Unless indicated otherwise, “FIX protein” or “FIX polypeptide” herein refers to the weight of the FIX portion (e.g., as defined in SEQ ID NO: 9) in the protein/polypeptide, i.e., excluding the weight of any additional portions such as fusion partners (e.g., albumin).

The terms “administration” or “administering” or “administered” are used interchangeably herein. Unless specifically stated otherwise the term administration refers to administration into a soft tissue.

The terms “treatment”, “therapy” and “treating” are used interchangeably herein and refer to therapeutic measures that cure, slow down, lessen symptoms of, and/or halt progression of a diagnosed pathologic condition or disorder. The terms “treatment”, “therapy” and “treating” may include prophylaxis, unless indicated otherwise. The terms “treatment”, “therapy” and “treating” also include on-demand treatment. A disorder is treated or prevented if administration of a Factor IX variant polypeptide as described herein to a subject (e.g., a human with Factor IX deficiency such as hemophilia B) results in a therapeutic or prophylactic effect. This means that the plasma level of Factor IX activity in the subject is increased following treatment, at least temporarily, when measured with at least one Factor IX assay. The Factor IX activity can be determined using an in vitro aPTT-based one stage clotting assay (ref 5 and 6) or a tail clip model (e.g., as described in the Examples). The increase may be clinically relevant, e.g., a reduction in the frequency or intensity of bleeding events.

By a “therapeutically effective amount” it is meant that the administration of that amount of Factor IX variant polypeptide to a subject, either in a single dose or as part of a series, is effective for treatment. By a “prophylactically effective amount” it is meant that the administration of that amount of Factor IX variant polypeptide to a subject, either in a single dose or as part of a series, is effective for prevention. Such methods have efficacy in the treating or preventing disorders where a pro-coagulant activity is needed (e.g., to prevent, reduce or inhibit bleeding) and include, without limitation, hemophilia, particularly hemophilia B.

The term “reduced binding” or “decreased binding” refers to Factor IX variant polypeptides that have reduced FIX binding to extracellular matrix compared to wild-type FIX, and includes FIX variants that exhibit no binding to extracellular matrix. FIX binding to extracellular matrix can be determined by various known biological assays, for example the competitor binding assay as described in (ref 4).

For the avoidance of any doubt, FIX variant polypeptides with reduced binding for use in the invention retain FIX clotting activity, e.g., they have the clotting activity of wild-type FIX, or they may even have a higher clotting activity than wild-type FIX. Clotting activity may be assessed by assays known in the art. Any reference to a method for treatment comprising administering FIX variant polypeptide to a subject, also covers the FIX variant polypeptide for use in said method for treatment, as well as the use of the FIX variant polypeptide in said method for treatment, and the use of the FIX variant polypeptide in the manufacture of a medicament for treating a disease.

The term “subject” refers to any animal (e.g., a mammal), including, but not limited to, humans, non-human primates, canines, felines, rabbits, rodents, and the like, which is to be the recipient of a particular treatment. The subject is preferably a human. Typically, the terms “subject” and “patient” are used interchangeably herein in reference to a human subject.

The term “pharmaceutically acceptable” refers to a substance approved or approvable by a regulatory agency of the Federal government or a state government or listed in the U.S. Pharmacopeia or other generally recognized pharmacopeia for use in animals, including humans.

The terms “pharmaceutically acceptable excipient, carrier, or adjuvant” or “acceptable pharmaceutical carrier” refer to an excipient, carrier, or adjuvant that can be administered to a patient, together with at least one agent of the present disclosure, and which does not destroy the pharmacological activity thereof and is non-toxic when administered in doses sufficient to deliver a therapeutic effect. In general, those of skill in the art and the U.S. FDA consider a pharmaceutically acceptable excipient, carrier, or adjuvant to be an inactive ingredient of any formulation.

The term “substantially pure” refers to a preparation comprising at least 75% by weight of Factor IX variant polypeptide, particularly at least 80% by weight, at least 85% by weight, at least 90% by weight, at least 95% by weight, or at least 96%, 97%, 98%, or 99% by weight, e.g., 90-99% or more by weight of Factor IX variant polypeptide. Purity may be measured by methods appropriate for the compound of interest (e.g., chromatographic methods, polyacrylamide gel electrophoresis, HPLC analysis, and the like).

The term “comprising” encompasses “including” as well as “consisting”, “consisting of” and/or “consisting essentially of”, e.g., a composition “comprising” X may consist exclusively of X or may include something additional, e.g., X+Y. It is also understood that wherever embodiments are described herein with the language “consisting essentially of” otherwise analogous embodiments described in terms of “consisting of” are also provided.

The term “about” in relation to a numerical value x is optional and means, for example, x±10%.

The word “substantially” does not exclude “completely”, e.g., a composition which is “substantially free” from Y may be completely free from Y. Where necessary, the word “substantially” may be omitted from the definition of the invention.

The term “and/or”, e.g., “X and/or Y” shall be understood to mean either “X and Y” or “X or Y” and shall be taken to provide explicit support for both meanings or for either meaning.

As used herein, the verb “to comprise” and its conjugations is used in its non-limiting sense to mean that items following the word are included, but items not specifically mentioned are not excluded. In addition, the verb “to consist” may be replaced, if necessary, by “to consist essentially of” meaning that a product as defined herein may comprise additional component(s) than the ones specifically identified, said additional component(s) not altering the unique characteristic of the invention.

Unless specifically stated, a process or method comprising numerous steps may comprise additional steps at the beginning or end of the method, or may comprise additional intervening steps. Also, steps may be combined, omitted or performed in an alternative order, if appropriate.

As used in the present disclosure and claims, the singular forms “a”, “an” and “the” include plural forms unless the context clearly dictates otherwise.

All patent and literature references cited in the present specification are hereby incorporated by reference in their entirety.

Various embodiments of the invention are described herein. It will be appreciated that the features specified in each embodiment may be combined with other specified features, to provide further embodiments. In particular, embodiments highlighted herein as being suitable, typical or preferred may be combined with each other (except when they are mutually exclusive).

### Factor IX (FIX) Variant Polypeptides

The invention relates to the use of FIX variant polypeptides that have decreased binding to extracellular matrix relative to wild-type FIX, for use in therapy by administering the FIX variant polypeptide to soft tissue (e.g., subcutaneous tissue).

### Extravascular FIX

The notion of extravascular FIX was first reported in 1983 when it was shown that FIX can bind to endothelial cells [ref 7]. Later in 1987, Stern et al. demonstrated that large amounts of FIX can exist in the extravascular space and that a rapid, reversible equilibrium exists between plasma and extravascular FIX (ref 8). Later studies demonstrated the direct binding of FIX to endothelial cells. In vitro experiments show that the zymogen form of FIX binds reversibly to vascular endothelium (refs 9, 10 and 11) and possibly to platelets (ref 12).

Experiments in hemophilia B (HB) mice have shown that FIX can occupy extravascular reservoirs and convey hemostatic protection for more than seven days while being undetected in plasma (ref 13). This study also estimated that there is significantly more FIX contained in these extravascular reservoirs than in circulation. In an attempt to characterize the phenomenon of the extravascular FIX reservoir, Cheung et al mutated the vitamin K-dependent γ-carboxyglutamic acid (Gla) domain of FIX at residue 5 (lysine) or residue 10 (valine), which reportedly strongly affected their interaction with endothelial cells. Specifically, a single point mutation of Lysine to Alanine (FIXK5A) or to Arginine (FIXK5R) at residue 5 of the FIX molecule resulted in altered endothelial cell binding affinity (ref 3). The FIXK5R variant was shown to have higher binding affinity to endothelial cells than wildtype FIX (FIXWT) in vitro, while the FIXK5A variant failed to bind bovine endothelial cells but retained normal clotting activity. In a subsequent study (ref 4), Cheung et al postulated that extracellular matrix, and possibly specifically collagen IV, was the FIX binding site on endothelial cells. Subsequent in vivo studies in HB mice found that HB mice infused with FIXK5R provided better hemostatic protection than wild-type FIX in a saphenous vein bleeding model. In contrast, HB mice infused with FIXK5A showed reduced clotting (ref 14). On this basis, the authors proposed that collagen IV binding by FIX provides a longer-lasting extravascular reservoir of FIX and therefore better hemostatic protection (see also refs 15 and 16).

It was therefore not obvious from these studies that FIX variant polypeptides that have decreased binding to extracellular matrix, such as the K5A variant, could be useful for treating bleeding disorders, let alone that they could provide increased hemostatic efficacy when administered to soft tissues. The inventors realised that when FIX is to be administered specifically to soft tissue (e.g., subcutaneously), FIX variant polypeptides that have decreased binding to extracellular matrix in fact provide increased hemostatic protection. For instance, the examples demonstrate that FIX variant polypeptides that have decreased binding to extracellular matrix (e.g., the K5A variant) have a higher hemostatic efficacy after subcutaneous administration, as compared to wild-type FIX. Without wishing to be bound by any particular theory, it is hypothesized that this higher hemostatic efficacy after subcutaneous administration is due to the fact that these FIX variant polypeptides can be released more easily into plasma circulation following subcutaneous administration, as they interact less strongly with extracellular matrix present at the administration site, such as collagen IV, in the extravascular space. Surprisingly, the absence of an extravascular reservoir of bound FIX does not appear to negatively affect hemostatic efficacy of these FIX variants such as the K5A variant when they are administered subcutaneously, in contrast to the effects previously described for those variants with other routes of administration (e.g., intravenous).

FIX variant polypeptides for use in the invention therefore have decreased binding to extracellular matrix, such as collagen IV. Examples of FIX variant polypeptides that have decreased binding for use in the invention include a FIX variant polypeptide comprising the amino acid alanine at a position corresponding to position 5 of wild-type Factor IX, a FIX variant polypeptide comprising the amino acid lysine at a position corresponding to position 10 of wild-type Factor IX, or more generally a FIX variant polypeptide comprising an amino acid with any hydrophobic or uncharged side chain at a position corresponding to position 5 of wild-type Factor IX, or a FIX variant polypeptide comprising an amino acid with a positively charged side chain at a position corresponding to position 10 of wild-type Factor IX, as long as they retain FIX clotting activity, e.g., they have the clotting activity of wild-type FIX, or they may even have a higher clotting activity than wild-type FIX; clotting activity can be measured by standard assays known to those skilled in the art. Amino acids that comprise a hydrophobic side chain (at pH 7) include Alanine, Valine, Isoleucine, Leucine, Methionine, Phenylalanine, Tyrosine and Tryptophan. Amino acids that comprise an uncharged side chain (at pH 7) include Serine, Threonine, Asparagine and Glutamine. Amino acids that comprise a positively charged side chain (at pH 7) include Lysine, Arginine and Histidine. In preferred embodiments, the FIX variant polypeptide comprises the amino acid alanine at a position corresponding to position 5 of wild-type Factor IX.

In some embodiments, the FIX variant polypeptide comprises the amino acid alanine at a position corresponding to position 5 of wild-type Factor IX but does not comprise the amino acid lysine at a position corresponding to position 10 of wild-type Factor IX (valine may instead be used at position 10).

The FIX variant polypeptide for use in the invention can also comprise two or more mutations (e.g., at positions 5 and 10) that decrease the binding of the polypeptide to extracellular matrix. For example, in some embodiments, the FIX variant polypeptide comprises the amino acid alanine at a position corresponding to position 5 of wild-type Factor IX and the amino acid lysine at a position corresponding to position 10 of wild-type Factor IX. In other embodiments, the FIX variant polypeptide comprises an amino acid with a hydrophobic or uncharged side chain at a position corresponding to position 5 of wild-type Factor IX and a positively charged side chain at a position corresponding to position 10 of wild-type Factor IX.

In one aspect, the invention therefore provides a Factor IX (FIX) variant polypeptide for use in a method of treating or preventing a disease or disorder comprising administering the FIX variant polypeptide to a soft tissue, wherein the FIX variant polypeptide comprises the amino acid alanine at a position corresponding to position 5 of wild-type Factor IX.

The invention also provides a method of treating or preventing a disease or disorder in a subject comprising administering a therapeutically or prophylactically effective amount of a FIX variant polypeptide to a soft tissue in the subject, wherein the FIX variant polypeptide comprises the amino acid alanine at a position corresponding to position 5 of wild-type Factor IX.

The invention further provides a use of a FIX variant polypeptide in the manufacture of a medicament for treating or preventing a disease or disorder in a subject, wherein the FIX variant polypeptide is to be administered to a soft tissue in the subject and wherein the FIX variant polypeptide comprises the amino acid alanine at a position corresponding to position 5 of wild-type Factor IX.

The invention further provides a FIX variant polypeptide for treating or preventing a disease or disorder, wherein the FIX variant polypeptide is to be administered to a soft tissue in the subject and wherein the FIX variant polypeptide comprises the amino acid alanine at a position corresponding to position 5 of wild-type Factor IX.

### Additional Factor IX Mutations

In further embodiments the FIX variant polypeptides for use in the invention can also comprise further mutations compared to wild-type Factor IX that can increase coagulation activity (e.g., increase specific activity) relative to wild-type Factor IX. Such a variant polypeptide is also referred to herein as a ‘high-activity’ FIX polypeptide, or a high-activity FIX variant polypeptide. Other terms are used in the art synonymously, e.g., ‘hyperactive’ FIX variants. These variants have the biological function of a Factor IX, i.e., the variant is able to generate Factor Xa, optionally after the Factor IX variant polypeptide has been converted to its active form (Factor IXa) by excision of the activation peptide. The variant is able to generate Factor Xa with a higher activity than wild-type FIX. Activation cleavage of Factor IX can be achieved in vitro, e.g., by Factor XIa or Factor VIIa/TF. Suitable in vitro assays to measure Factor IX activity are known to the person skilled in the art (e.g., one-stage clotting assay such as an aPTT assay, chromogenic assay, etc.).

An exemplary high-activity Factor IX variant polypeptide comprises leucine (L) at a position corresponding to position 338 of wild-type Factor IX, which typically has an arginine (R) at that position (“R338L”). One such exemplary polypeptide is the ‘Padua’ mutant, described in ref 17. See SEQ ID NO: 10. The specific activity of the ‘Padua’ mutant is typically at least around 5-8 fold higher compared to wild-type Factor IX.

Therefore, in some embodiments, the Factor IX (FIX) variant polypeptides for use in the invention comprise the amino acid alanine at a position corresponding to position 5 of wild-type Factor IX and a leucine at a position corresponding to position 338 of wild-type Factor IX. Other exemplary high-activity Factor IX variants are E410H, E410K, R338V, and R338L+E410K, and those described in ref. 18, e.g., comprising the amino acid H at a position corresponding to position 410 of wild-type Factor IX, and comprising an amino acid other than R at a position corresponding to position 338 of wild-type Factor IX, for example comprising an amino acid selected from the group consisting of V, T and W at a position corresponding to position 338 of wild-type Factor IX, e.g., R338V+E410H, R338T+E410H, R338W+E410H, and R338L+E410H. Another useful variant is R318Y+R338E+T343R.

Therefore, in some embodiments, the FIX variant polypeptides for use in the invention comprise the amino acid alanine at a position corresponding to position 5 of wild-type Factor IX, an amino acid selected from valine, threonine and tryptophan at a position corresponding to position 338 of wild-type Factor IX and the amino acid histidine at a position corresponding to position 410 of wild-type Factor IX. In a specific embodiment, the FIX variant polypeptides for use in the invention comprise the amino acid alanine at a position corresponding to position 5 of wild-type Factor IX, the amino acid valine at a position corresponding to position 338 of wild-type Factor IX and the amino acid histidine at a position corresponding to position 410 of wild-type Factor IX.

As mentioned above, a further high-activity Factor IX variant for use in the invention is the Dalcinonacog alfa variant (also known as CB 2679d), see SEQ ID NO: 19. Dalcinonacog alfa has three amino acid substitutions in two loops within the FIX protein. Based on mature FIX sequence numbering, (1) R318Y located in the ‘150-loop’, stabilizes activated FIX (FIXa), directly interacts with the substrate factor X (FX) and provides resistance to antithrombin; (2) R338E, and (3) T343R, both located in the ‘170-loop’, significantly enhance affinity to the cofactor, activated factor VIII (FVIIIa) and increase the catalytic activity FIXa. R318Y/R338E/T343R refer to R150Y/R170E/T175R in classic chymotrypsin numbering [ref 19] and R364Y/R384E/T389R in the Human Genome Variation Society (HGVS) nomenclature, which includes the 46 amino acid propeptide [ref 20]. Therefore, in some embodiments, the FIX variant polypeptides for use in the invention comprise the amino acid alanine at a position corresponding to position 5 of wild-type Factor IX, the amino acid tyrosine at a position corresponding to position 318 of wild-type Factor IX, the amino acid glutamic acid at a position corresponding to position 338 of wild-type Factor IX and the amino acid arginine at a position corresponding to position 343 of wild-type Factor IX.

Further exemplary high-activity Factor IX variant polypeptides include those listed in Table 1 below. (ref 21).

TABLE 1

| | Exemplary fold change | |
|---|---|---|
| Amino acid | in FIX activity relative | |
| substitution(s) | to FIX-WT | Reference(s) |
| E410H | 4.6 | ref. 22 |
| G4Y | 1.2 | ref. 23 |
| V10K | 1.6 | ref. 23, ref. 3 |
| V86A | 1.1 | ref. 24 |
| K265T | 1.9 | ref. 25, ref. 26 |
| E277A | 1.3 | ref. 24 |
| N346A | 1.2 | ref. 27 |
| S377W | 1.4 | ref. 23 |
| R338L + S377W | 12 | ref. 23 |
| V10K + R338L + S337W | 19 | ref. 23 |
| R318Y + R338E + T343R | 17 | ref. 28 |
| V86A + E277A + R338L | 22 | ref. 29 |
| G4Y + V86A + R338L + | 15 | ref. 23 |
| S337W | | |

The numbering in Table 1 refers to the positions in the mature FIX protein without propeptide sequence (SEQ ID NO: 1). The activity was determined with a one-stage clotting assay.

The skilled person is able to identify and verify these and other high-activity Factor IX variant polypeptides, by determining the specific (molar) activity of a Factor IX polypeptide using methods known in the art, and comparing that activity with wild-type Factor IX.

The Factor IX variant polypeptide can be derived from a Factor IX polypeptide sequence of any mammalian species. In a particular embodiment, the Factor IX variant polypeptide is derived from a Factor IX polypeptide sequence of human origin. Gene ID: 2158 (https://www.ncbi.nlm.nih.gov/gene/2158), GenBank Accession Nos. NM_000133.3 (https://www.ncbi.nlm.nih.gov/nuccore/NM_000133.3), NP 000124.1 (https://www.ncbi.nlm.nih.gov/protein/NP_000124.1?report=genpept), and UniProt entry P00740 (https://www.uniprot.org/uniprot/P00740) provide examples of the amino acid and/or nucleotide sequences of wild-type human Factor IX.

The Factor IX variant polypeptide according to the invention may be derived from mature (i.e., excluding signal peptide and propeptide) wild-type Factor IX, for example of human origin, the amino acid sequence of which is shown in SEQ ID NO: 1. That polypeptide sequence is ‘isoform 1’ of human Factor IX.

### Administration Routes

The examples demonstrate that FIX variant polypeptides comprising the amino acid alanine at a position corresponding to position 5 of wild-type Factor IX are more hemostatically effective than wild type FIX when administered into subcutaneous tissue. Without wishing to be bound by any particular theory, it is hypothesized that the reason that these FIX variant polypeptides are more hemostatically effective after subcutaneous administration is because they bind less strongly to the extracellular matrix and are therefore released from the extracellular space into circulation more quickly. Based on these data, it is therefore plausible that the disclosed FIX variant polypeptides would also be more hemostatically effective compared to wild type FIX when administered into soft tissues more generally.

Therefore, the Factor IX (FIX) variant polypeptides for use in the invention are administered to a soft tissue. The term is understood by those skilled in the art. For instance, soft tissue administration is defined by the FDA as administration into any soft tissue (https://www.fda.gov/drugs/data-standards-manual-monographs/route-administration). Soft tissue is any tissue in the body that is not hardened by the processes of ossification or calcification such as bones and teeth. In one embodiment, the soft tissue excludes muscle tissue. In another embodiment, the soft tissue excludes liver tissue. In preferred embodiments, the soft tissue that the FIX variant polypeptide is administered into is skin tissue (including subcutaneous tissue) or mucosal tissue (including gastrointestinal mucosal tissue).

The FIX variant polypeptides are for administration to a subject, such as an animal, typically a human subject.

The methods and uses described herein do not involve the intravenous administration of FIX variant polypeptides. For example, the invention provides a Factor IX (FIX) variant polypeptide for use in a method of treating or preventing a disease (e.g., a bleeding disorder), wherein the FIX variant polypeptide comprises the amino acid alanine at a position corresponding to position 5 of wild-type Factor IX, wherein the FIX variant polypeptide is not administered intravenously.

In some embodiments, the methods and uses described herein do not involve the intramuscular administration of FIX variant polypeptides.

Administration into a soft tissue immediately exposes the FIX variant polypeptides to components in the extracellular space (as known as the interstitial space) between cells. For example, following administration of FIX variant polypeptides to soft tissue, at least a portion of the FIX variant polypeptides is delivered directly into the extracellular space, and another portion of the FIX variant polypeptides may be delivered into or taken up by cells and then the FIX variant polypeptides are secreted out of the cells into the extracellular space. The methods and uses described herein are therefore distinct from gene-based (e.g., viral or non-viral vectors) approaches wherein a nucleic acid sequence encoding FIX is administered (e.g., to muscle tissue) and the FIX polypeptide is produced intracellularly.

In a preferred embodiment, the soft tissue is skin tissue. For the purposes of this disclosure, the skin comprises three main layers—the hypodermis (subcutaneous tissue) is the innermost layer of skin; the dermis is the middle layer, and the epidermis is the outermost layer. Subcutaneous administration (e.g., subcutaneous injection) refers to the administration of a substance into the hypodermis. For the avoidance of any doubt, references to “administration into the skin” or “administration into skin tissue”, in the context of this disclosure, encompass subcutaneous administration (administration (into the hypodermis). Furthermore, subcutaneous administration that is characterised as administration “under” or “beneath” or “underneath” the skin (or synonymous terms), is also encompassed by the present invention.

In some embodiments therefore, the FIX variant polypeptides are administered into skin tissue. In some embodiments, the FIX variant polypeptides are administered into subcutaneous tissue (hypodermal tissue), into dermal tissue or into epidermal tissue. Accordingly, the administration can be subcutaneous, intradermal, topical (e.g., epicutaneous) or transdermal (e.g., via transdermal injection or absorption). In preferred embodiments, the FIX variant polypeptides are administered subcutaneously.

In preferred embodiments, the Factor IX (FIX) variant polypeptide for use in the invention comprises the amino acid alanine at a position corresponding to position 5 of wild-type Factor IX and is administered subcutaneously. For example, the invention provides a method of treating or preventing a disease in a subject comprising subcutaneously administering to the subject an effective amount of a FIX variant polypeptide comprising the amino acid alanine at a position corresponding to position 5 of wild-type Factor IX.

The invention also provides a Factor IX (FIX) variant polypeptide for use in a method of treating or preventing a disease in a subject comprising subcutaneously administering the FIX variant polypeptide, wherein the FIX variant polypeptide comprises the amino acid alanine at a position corresponding to position 5 of wild-type Factor IX.

Also provided is a use of a Factor IX variant polypeptide in the manufacture of a medicament for treating or preventing a disease in a subject, wherein the FIX variant polypeptide is to be administered subcutaneously to the subject and wherein the FIX variant polypeptide comprises the amino acid alanine at a position corresponding to position 5 of wild-type Factor IX.

The invention also provides a use of a FIX variant polypeptide for treating or preventing a disease in a subject comprising subcutaneously administering the FIX variant polypeptide to the subject, wherein the FIX variant polypeptide comprises the amino acid alanine at a position corresponding to position 5 of wild-type Factor IX.

In some embodiments, the FIX variant polypeptides are administered into mucosal tissue, such as gastrointestinal mucosal tissue. In some embodiments, the FIX variant polypeptides are administered enterally (via the human gastrointestinal tract). Examples of enteral administration include oral, sublingual, gastric, and rectal administration.

The FIX variant polypeptides can be administered by injection into the mucosal tissue of the gastrointestinal tract using a drug delivery device (also known as an applicator) that can be taken orally, which autonomously positions itself to engage with and inject a drug into the GI tissue. Exemplary drug delivery devices are described in ref 30. Exemplary devices include SOMA (self-orienting millimeter-scale applicator) (ref 31), BIONDD™ (ref 32) and RaniPill™ (ref 33 and 34). In some embodiments, the FIX variant polypeptides are administered by injection into the mucosal tissue in the stomach, for example using a BIONDD™ device. BIONDD™ is designed to insert a drug-loaded biodegradable spike to the stomach wall. It consists of a capsule that attaches and delivers a drug to the stomach tissue.

### Bleeding Disorders

In a preferred embodiment, the Factor IX variant polypeptides described herein are for treating or preventing a bleeding disorder. The bleeding disorder may be any disorder which requires pro-coagulant (e.g., to prevent, reduce or inhibit bleeding). An exemplary bleeding disorder is hemophilia, particularly hemophilia B.

The invention therefore provides FIX variant polypeptide for use in a method of treating or preventing a bleeding disorder comprising administering the FIX variant polypeptide to a soft tissue, wherein the FIX variant polypeptide comprises the amino acid alanine at a position corresponding to position 5 of wild-type Factor IX.

The invention also provides a method of treating or preventing a bleeding disorder in a subject comprising administering a therapeutically or prophylactically effective amount of a FIX variant polypeptide to a soft tissue in the subject, wherein the FIX variant polypeptide comprises the amino acid alanine at a position corresponding to position 5 of wild-type Factor IX.

The invention further provides a use of a FIX variant polypeptide in the manufacture of a medicament for treating or preventing a bleeding disorder in a subject, wherein the FIX variant polypeptide is to be administered to a soft tissue in the subject and wherein the FIX variant polypeptide comprises the amino acid alanine at a position corresponding to position 5 of wild-type Factor IX.

The invention further provides a FIX variant polypeptide for treating or preventing a bleeding disorder, wherein the FIX variant polypeptide is to be administered to a soft tissue in the subject and wherein the FIX variant polypeptide comprises the amino acid alanine at a position corresponding to position 5 of wild-type Factor IX.

The treating or preventing may include on-demand control of bleeding episodes, perioperative management of bleeding, and/or routine prophylaxis to prevent or reduce the frequency of bleeding episodes. For example, treatment may include on-demand control of bleeding episodes or perioperative management of bleeding. Prevention may include prevention of bleeding episodes or reducing the frequency of bleeding episodes.

The subject is typically a human. The subject may be an adult or a child. The subject may have a basal (without prophylaxis or treatment) plasma Factor IX activity of 40% or less, 30% or less, 20% or less, 10% or less, 5% or less, 4% or less, 3% or less, 2% or less, between 1-5%, or 1% or less, compared to the plasma Factor IX activity of a healthy subject. In a particular embodiment, the subject is a paediatric subject (a child), e.g., 18 years or younger. In one embodiment, the subject is not eligible to receive FIX gene therapy.

In some embodiments, the FIX variant polypeptide is administered at a dose of 20 IU/kg to 350 IU/kg. In certain embodiments, the FIX variant polypeptide is administered at a dose of IU/kg to 300 IU/kg, 30 IU/kg to 250 IU/kg, 50 IU/kg to 200 IU/kg or 50 IU/kg to 150 IU/kg. In some embodiments, the FIX variant polypeptide is administered at a dose of about 25 IU/kg, IU/kg, 50 IU/kg, 75 IU/kg, 100 IU/kg, 150 IU/kg, 200 IU/kg, 250 IU/kg, 300 IU/kg or 350 IU/kg. In certain embodiments, the FIX variant polypeptide is administered at a dose of about 50 IU/kg, 100 IU/kg or 150 IU/kg.

In one embodiment, the FIX polypeptide is administered in a composition that does not contain anti-thrombotic substances (e.g., heparin).

Bleeding disorders include hemophilia (hemophilia A, hemophilia B, hemophilia A and B patients with inhibitory antibodies; in particular hemophilia B), deficiencies in at least one coagulation factor (e.g., Factors VII, IX, X, XI, V, XII, II, and/or von Willebrand factor; in particular Factor IX), combined FV/FVIII deficiency, vitamin K epoxide reductase CI deficiency, gamma-carboxylase deficiency; bleeding associated with trauma, injury, thrombosis, thrombocytopenia, stroke, coagulopathy (hypocoagulability), disseminated intravascular coagulation (DIC); over-anticoagulation associated with heparin, low molecular weight heparin, pentasaccharide, warfarin, small molecule antithrombotics (i.e., FXa inhibitors); and platelet disorders such as, Bernard Soulier syndrome, Glanzman thromblastemia, and storage pool deficiency.

In a preferred embodiment, the method or use described above is for treatment or prevention of bleeding in a subject with hemophilia B, which is also known in the art as congenital factor IX deficiency.

One way of expressing Factor IX activity in plasma is as a percentage relative to normal human plasma. Another way of expressing Factor IX activity in plasma is in International Units (IU) relative to an International Standard for Factor IX in plasma. One IU of Factor IX activity in plasma is equivalent to that quantity of Factor IX in one mL of normal human plasma.

One way of checking efficacy of prophylaxis or treatment is by measuring the plasma Factor IX activity in the subject after prophylaxis or treatment, and comparing it to the plasma Factor IX activity in that subject before prophylaxis or treatment. An increase in Factor IX activity after prophylaxis or treatment (e.g., from <1%, or 1%-5%, or 5-40% of normal human plasma to e.g., 15%, 20%, >25%, >30%, >35%, >40%, >50%, or >60% peak levels of normal human plasma, e.g., from <5% to >5% such as to 5-40%) indicates a prophylactic or therapeutic effect. Factor IX levels of 5-10% of normal human serum have been targeted in clinical trials for achieving bleeding control while on prophylaxis.

A prophylactic or therapeutic effect is also achieved where the Factor IX activity after prophylaxis or treatment is sufficient to prevent, reduce or inhibit bleeding.

The Factor IX activity after prophylaxis or treatment may results in troughs of at least 15-40%, or may even be outside of the pathological range (e.g., >40% peak levels of normal human serum).

Factor IX activity can be measured using any Factor IX activity assay known to the skilled person, for example using an aPTT assay (a decrease in aPTT value indicates increased Factor IX activity). In a preferred embodiment therefore Factor IX activity is determined using an in vitro aPTT-based one stage clotting assay [ref 5 and 6].

A Factor IX variant polypeptide for use in the invention may have a higher specific molar activity when administered in vivo to a subject than the corresponding wild-type Factor IX polypeptide.

Such high-activity variants are described above. For example, the % increase in plasma Factor IX activity (e.g., measured using an in vitro aPTT-based one stage clotting assay) may be higher with a Factor IX variant polypeptide as described herein as compared with using the same molar amount of the corresponding wild-type Factor IX polypeptide. Another way of describing this is that the aPTT time in a serum sample after administering a Factor IX variant polypeptide as described herein is shorter as compared with the same molar amount of the corresponding wild-type Factor IX polypeptide.

### Preparing a Factor IX Variant Polypeptide

A Factor IX variant polypeptide for use in the invention can be made using standard techniques well known to the skilled person in the art. For example, the cDNA sequence of a wild-type Factor IX (e.g., SEQ ID NO: 2) may be modified using standard mutagenesis techniques (e.g., site-directed mutagenesis) so that it encodes the desired Factor IX variant polypeptide, e.g., encoding the amino acid alanine at a position corresponding to position 5 of wild-type Factor IX (which encodes lysine (K) at that position). An N-terminal leader peptide for the purposes of recombinant protein production can be used, based on the natural Factor IX leader peptide (as shown in SEQ ID NO: 3) or alternatives known to the skilled person in the art. The cDNA sequence may be inserted into a suitable expression plasmid to express the recombinant Factor IX variant polypeptide. This is typically performed using mammalian cells (e.g., HEK for transient expression or a CHO cell line for stable expression), although other types of cells that can produce glycosylated and correctly folded proteins can also be used. The recombinant Factor IX variant polypeptide may subsequently be purified, for example using anion exchange chromatography.

The Factor IX variant polypeptide may be combined with other agents and/or with a pharmaceutically acceptable carrier.

### Fusions and Conjugates

A Factor IX variant polypeptide for use in the invention can also be provided as part of a fusion with another moiety, e.g., with an albumin (for example attached via a cleavable linker).

The Factor IX variant polypeptide may be provided in fusion with, or it may be conjugated to, one or more additional portions. The one or more additional portions are typically different from Factor IX, i.e., they do not have the biological function of Factor IX as defined above (they do not have the ability to generate Factor Xa). This means that fragments of Factor IX, e.g., linkers comprising a fragment of a Factor IX-derived polypeptide sequence, but which do not on their own have the function of Factor IX, may be such “one or more additional portions”, i.e., they are not part of the Factor IX portion but they may be part of the molecule that comprises the Factor IX portion.

### Half-Life Enhancing Portion and Linker

In an exemplary embodiment, the FIX variant polypeptide is linked to a half-life enhancing portion. The half-life enhancing portion may comprise one or more polypeptides (half-life enhancing polypeptides, HLEPs). In one embodiment, the HLEP is albumin, e.g., recombinant human albumin. In another embodiment, the HLEP is a fragment of an antibody (immunoglobulin), such as the Fc fragment, e.g., IgG Fc, such as IgG1 Fc. Alternatively, the HLEP may be a C-terminal peptide of human chorionic gonadotropin (CTP). The HLEP may also be an unstructured recombinant polypeptide (e.g., XTEN). Such molecules are also referred to in the art as fusion polypeptides.

The FIX variant polypeptide may be linked to the HLEP via a cleavable linker, in particular a cleavable peptide linker. Typically, the cleavable linker is cleavable by the same protease that activates Factor IX. Such cleavable linkers therefore provide a high molar specific activity of the fusion polypeptide.

The FIX variant polypeptide may also be PEGylated, i.e., one or more polyethylene glycol moieties are conjugated to the FIX variant polypeptide, using methods known in the art.

A FIX variant polypeptide for use in the invention may comprise one half-life enhancing portion, or more than one half-life enhancing portions. The wording “a half-life enhancing portion” therefore covers one or more half-life enhancing portions. The half-life enhancing portions may be of the same type. The half-life enhancing portions may be of different types. For example, the FIX variant polypeptide may be linked to XTEN (e.g., XTEN72) and additionally to an Fc domain (e.g., human IgG1 Fc).

Preferably, the half-life enhancing portion is capable of extending the half-life of the FIX variant polypeptide in vivo (in plasma) by at least about 25% as compared to the non-fused FIX variant polypeptide. Preferably, the half-life enhancing portion is capable of extending the half-life of the FIX variant polypeptide in vivo (in plasma) by at least about 50%, and more preferably by more than 100%. The in vivo half-life is generally determined as the terminal half-life or the β-half-life.

### Albumin

As used herein, “albumin” refers collectively to an albumin polypeptide or amino acid sequence, or an albumin fragment, variant or analog having one or more functional activities (biological activities) of albumin. In particular, “albumin” may refer to human albumin (HA) or a fragment thereof, especially the mature form of human albumin as shown in SEQ ID NO: 5 herein. The albumin may also be derived from other species, in particular other vertebrates. The albumin portion of the fusion polypeptide may comprise the full length of the HA sequence as described in SEQ ID NO: 5, or it may include one or more fragments thereof that are capable of stabilizing or prolonging the therapeutic activity of the Factor IX variant polypeptide. Such fragments may be of 10 or more amino acids in length or may include about 15, 20, 25, 30, 50, or more contiguous amino acids from the HA sequence or may include part or all of the specific domains of HA. These and other suitable albumin portions (including variants) are described in reference 36.

Structurally related family members of the albumin family may also be used as HLEPs. For example, alpha-fetopolypeptide (AFP, reference 35) is a member of the albumin family and may also be used to enhance the half-life of a Factor IX variant polypeptide. Such half-life enhancing polypeptides are described in reference 36. Another option is afamin (AFM, reference 37) or vitamin D binding polypeptide (DBP, reference 38). Fragments of these polypeptides may also be used.

In embodiments that use albumin HLEPs, the albumin is typically provided as a genetic fusion with the Factor IX portion. This means that a single cDNA molecule encodes the Factor IX portion and the albumin portion, optionally with an intervening sequence encoding a linker, such as a cleavable linker.

### Immunoglobulin

An immunoglobulin (lg) or a fragment thereof may also be used as a HLEP. An example of a suitable immunoglobulin is IgG, or an IgG-fragment, such as an Fc region. The Fc region may be an Fc domain (e.g., two polypeptide chains each of which comprises the hinge region (or part of the hinge region), the CH2 region and the CH3 region). Thus, a Factor IX variant polypeptide may be fused to an Fc domain, directly or via a linker. In embodiments that use a linker, the linker may be cleavable.

Monomers, dimers and hybrids are all encompassed. For example, the Factor IX variant polypeptide may be a heterodimer comprising two polypeptide chains, wherein the first chain comprises a Factor IX portion linked to the hinge region (or part of the hinge region), the CH2 region and the CH3 region of an immunoglobulin (e.g., IgG1), and the second chain comprises the hinge region (or part of the hinge region), the CH2 region and the CH3 region of an immunoglobulin (e.g., IgG1).

In another embodiment, the Factor IX variant polypeptide is a homodimer comprising two polypeptide chains, wherein each chain comprises a Factor IX portion linked to the hinge region (or part of the hinge region), the CH2 region and the CH3 region of an immunoglobulin (e.g., IgG1).

In a further embodiment, the Factor IX variant polypeptide is a monomer comprising a Factor IX portion linked to the hinge region (or part of the hinge region), the CH2 region and the CH3 region of an immunoglobulin (e.g., IgG1).

Other examples of suitable Factor IX IgG Fc fusion molecule configurations are found, e.g., in reference 39.

An exemplary Fc polypeptide (derived from the human IgG1 Fc domain) is shown in SEQ ID NO: 6. Another exemplary Fc polypeptide (derived from the human IgG1 Fc domain) is shown in SEQ ID NO: 7.

In any of these embodiments, the Factor IX portion may be linked directly or via a linker to the Fc portion. In embodiments that use a linker, the linker may be cleavable or non-cleavable. In particular embodiments, the linker is cleavable. An exemplary cleavable linker is shown in SEQ ID NO: 8.

An exemplary Fc portion is the Fc portion of Eftrenonacog alfa (Alprolix®). See also references 40, 41 or 42.

### C-Terminal Peptide of Human Chorionic Gonadotropin (CTP)

Another exemplary half-life enhancing portion is a C-terminal peptide of human chorionic gonadotropin (CTP). CTP is based on a natural peptide of 31 amino acids length, the C-terminal peptide of the beta chain of human chorionic gonadotropin (hCG).

One or more units of CTP can be fused to a Factor IX portion. The one or more units of CTP can be fused to the N-terminus and/or to the C-terminus of Factor IX, preferably to the C-terminus.

In one embodiment, the Factor IX variant polypeptide is a CTP-modified Factor IX comprising a Factor IX variant polypeptide as described herein linked with three to five CTPs, optionally wherein the CTPs are attached to the C-terminus of the Factor IX variant polypeptide. In a specific embodiment, three tandem units of CTP are attached the Factor IX variant polypeptide, optionally at the C-terminus of the Factor IX variant polypeptide.

In any of these embodiments, at least one of the CTP may be attached to the Factor IX portion via a linker. The linker may be a peptide bond. The linker may be cleavable.

In an exemplary embodiment, the CTP sequence comprises SEQ ID NO: 11. In another exemplary embodiment, the CTP sequence comprises SEQ ID NO: 12. In another exemplary embodiment, the CTP sequence comprises SEQ ID NO: 13.

Other suitable CTP sequences and related methods are known to the skilled person in the art, e.g., see references 43, 44 or 45.

### Unstructured Recombinant Polypeptide

Another exemplary half-life enhancing portion is an unstructured recombinant polypeptide. An example of such an unstructured recombinant polypeptide is XTEN, see, e.g., reference 46. In one embodiment, the Factor IX variant polypeptide is therefore a Factor IX variant polypeptide fused with at least one XTEN. XTEN may be fused to the Factor IX portion by insertion into the Factor IX variant polypeptide sequence while maintaining the biological activity of Factor IX. For example, the XTEN may be inserted between two neighbouring amino acids in the activation peptide of Factor IX at a position that does not prevent cleavage of the activation peptide during coagulation when XTEN is inserted. Alternatively, XTEN may fused to the C-terminus and/or N-terminus of the Factor IX, preferably the C-terminus. XTEN may be fused to the C-terminus and/or N-terminus (preferably C-terminus) of the Factor IX via a linker, e.g., a cleavable linker. The linker may be cleavable by thrombin.

A preferred XTEN is XTEN72. An exemplary XTEN72 sequence is shown in SEQ ID NO: 14. An alternative XTEN sequence is shown in SEQ ID NO: 15. Other suitable sequences and methods are disclosed in, e.g., references 47, 48 or 49.

In a specific embodiment, the Factor IX variant polypeptide comprises XTEN72 linked to the activation peptide of Factor IX and wherein the Factor IX portion is furthermore linked to a human IgG1 Fc domain at the C-terminus of the Factor IX portion.

### PEGylation

Another exemplary half-life enhancing portion is polyethylene glycol (PEG). GlycoPEGylation is within the scope of the term “PEGylation” as used herein. For example, a ca. 40 kDa PEG portion may be covalently attached to the Factor IX variant polypeptide, for example via a specific N-linked glycan within the activation peptide.

An example of a glycoPEG moiety is the glycoPEG moiety is nonacog beta pegol (Refixia®) (see also reference 50), in which an average of one non-reducing end of a glycan at N157 or N167 of Factor IX (numbering according to SEQ ID NO: 1) is attached to neuraminic acid conjugated to two PEG polymers (total average molecular weight of the polymers is ca. 42 kDa) via the amino group.

PEGylation of Factor IX polypeptide is also taught, for example, in references 51, 52 and 53.

### Linker

A Factor IX variant polypeptide comprising a half-life enhancing portion may employ a cleavable linker, in particular a proteolytically cleavable linker. The linker is generally positioned between the Factor IX polypeptide portion and a half-life enhancing portion. The linker may liberate the Factor IX portion upon cleavage of the linker by a protease of the coagulation cascade, e.g., a protease that is also capable of converting Factor IX to its activated form, e.g., FXIa or VIIa/tissue factor (TF). Cleavable linkers are particularly useful when the HLEP is albumin.

Although it is desirable to have an enhanced Factor IX in vivo half-life, it is desirable to limit the half-life of the Factor IX once it has been activated, to reduce the risk of a prothrombotic effect, especially with a hyperactive Factor IX variant polypeptide. In some embodiments therefore, a cleavable linker links the Factor IX variant polypeptide to a half-life enhancing portion, thereby providing a Factor IX variant polypeptide with a longer half-life relative to a non-fusion polypeptide. However, once bleeding occurs and the coagulation cascade has been initiated, a protease of the coagulation cascade activates the Factor IX variant polypeptide which has increased specific activity relative to, e.g., the corresponding wild-type Factor IX. At the same time, the linker is cleaved and the activated Factor IX variant polypeptide is liberated from the half-life enhancing portion, thereby reducing the risk of a prothrombotic effect due to any prolonged increased Factor IX activity.

The linker may be a fragment of Factor IX, preferably a fragment that is involved in Factor IX activation. For example, the linker may comprise such a fragment of a Factor IX sequence, extended by an N-terminal residue, such as a proline residue. An exemplary cleavable linker is shown in SEQ ID NO: 8. Other cleavable linkers are described in reference 36.

A Factor IX variant polypeptide linked to a half-life enhancing portion via an intervening cleavable linker may have at least 25% higher molar specific activity compared to the corresponding molecule with a non-cleavable linker (e.g., GGGGGGV, SEQ ID NO: 16), when measured in at least one coagulation-related assay, examples of which are known to the skilled person in the art, e.g., an aPTT one-stage assay. Preferably, a Factor IX variant polypeptide linked to a half-life enhancing portion via an intervening cleavable linker has at least 50%, more preferably at least 100% increased molar specific activity compared to the corresponding molecule without cleavable linker.

In one embodiment therefore, the FIX variant polypeptide for use in the invention comprises the amino acid alanine at a position corresponding to position 5 of wild-type Factor IX (and optionally one or more further mutations relative to wild-type FIX as described herein, to further reduce binding to extracellular matrix (e.g., V10K) and/or to increase the clotting activity of FIX (e.g., R338L)), wherein the FIX is linked to a half-life enhancing portion as described herein (e.g., an albumin), optionally via a cleavable linker as described herein.

### Pharmaceutical Compositions

The FIX variant polypeptides can be provided as a pharmaceutical composition. The pharmaceutical composition may be formulated with a pharmaceutically acceptable carrier. The invention therefore also provides a pharmaceutical composition comprising a Factor IX (FIX) variant polypeptide for use in a method of treating or preventing a bleeding disorder comprising administering the pharmaceutical composition to a soft tissue, wherein the FIX variant polypeptide comprises the amino acid alanine at a position corresponding to position 5 of wild-type Factor IX.

The invention also provides a method of treating or preventing a bleeding disorder in a subject comprising administering a therapeutically or prophylactically effective amount of a pharmaceutical composition comprising a FIX variant polypeptide to a soft tissue in the subject, wherein the FIX variant polypeptide comprises the amino acid alanine at a position corresponding to position 5 of wild-type Factor IX.

The invention further provides a use of a pharmaceutical composition comprising a FIX variant polypeptide in the manufacture of a medicament for treating or preventing a bleeding disorder in a subject, wherein the pharmaceutical composition is to be administered to a soft tissue in the subject and wherein the FIX variant polypeptide comprises the amino acid alanine at a position corresponding to position 5 of wild-type Factor IX.

The invention further provides a pharmaceutical composition comprising a FIX variant polypeptide for treating or preventing a bleeding disorder, wherein the pharmaceutical composition is to be administered to a soft tissue in the subject and wherein the FIX variant polypeptide comprises the amino acid alanine at a position corresponding to position 5 of wild-type Factor IX.

The pharmaceutical composition is for administration to a subject, such as an animal, typically a human subject.

The pharmaceutical composition is pharmaceutically acceptable and typically includes a suitable carrier. A thorough discussion of pharmaceutically acceptable carriers is available in reference 54. The composition is preferably sterile, pyrogen- and/or preservative-free.

Thus, the Factor IX variant polypeptide may be provided in buffered liquid form, e.g., in a citrate buffer, optionally containing a stabiliser and/or a bulking agent. An exemplary pharmaceutical composition for use in the invention comprises a Factor IX variant polypeptide, tri-sodium citrate dihydrate, polysorbate 80, mannitol, sucrose, hydrochloric acid, and sterile water. In an exemplary formulation, the components are 25 mM tri-sodium citrate dihydrate, 0.006%-0.024% polysorbate 80, 18-29 g/L mannitol, 7-12 g/L sucrose, hydrochloric acid for adjusting the pH to 6.6-7.2 (e.g., pH 6.8), and sterile water. In a preferred embodiment, the formulation is tri-sodium-citrate-2*H2O 30 mmol/L, D-mannitol 35.5 g/L, sucrose 14.0 g/L, polysorbate 80 0.00030 mL/L, pH 7.0.

Alternatively, the Factor IX variant polypeptide in the composition is lyophilized but is reconstituted with liquid diluent prior to administration, e.g., with sterile water for injection. Typical excipients in a composition comprising lyophilized Factor IX variant polypeptide include tri-sodium citrate dihydrate, polysorbate 80, mannitol, sucrose, and/or hydrochloric acid.

In some embodiments, the composition is suitable for administration to soft tissue, for example subcutaneous administration, optionally after reconstitution or dilution.

Compositions may be prophylactic (to prevent bleeding) or therapeutic (to treat bleeding).

### BRIEF DESCRIPTION OF THE DRAWINGS

FIG. 1: Pharmacokinetic profile of FIX variant polypeptides in HB mice after intravenous administration. Samples were collected at different timepoints until 336 hours post administration. LLOQ (<1.6 pmol/mL) refers to the lowest concentration of rFIX antigen that can be reliably detected based on ELISA. Values≤LLOQ were not plotted. Timepoints plotted: 0 (timepoint of the injection) and up to 168 hours. Each data point represents the mean±SD of n=3-5 mice and α indicates n=1. Only 1 out of 3 animals (α) had detectable levels in the rFIXK5R group at 48 hours and only 1 out of 5 animals (α) had detectable levels at 72 and 168 hours in the rFIXWT group.

FIG. 2: Pharmacokinetic profile of FIX variant polypeptides in HB mice after subcutaneous administration. Samples were collected at different timepoints until 336 hours post administration. LLOQ (<1.6 pmol/mL) refers to the lowest concentration of rFIX antigen that can be reliably detected based on ELISA. Values≤LLOQ were not plotted. Timepoints plotted: 0 (timepoint of the injection) and up to 72 hours. Each data point represents the mean±SD of n=3-5 mice and a indicates n=1. Only 1 out of 3 animals (a) had detectable levels in the rFIXK5R group at 24 hours and in the rFIXWT group at 72 hours.

FIG. 3: Livers from HB mice (n=3) following intravenous administration of rFIX (25 nmol/kg) or saline control. (A) Quantitative analysis (samples from each group were prepared and imaged in parallel under identical conditions) of FIX positive liver sections collected at 5 min (0.08 hr), 24, 72, and 120 hours after treatment with rFIX proteins was performed in ZEN Software. Each bar represents the median±95% CI of 2-3 sampled livers. The bars represent rFIXWT, rFIXK5A, rFIXK5R, respectively. The groups were compared at each timepoint using 1-way ANOVA test to determine the p-value. ns, not significant and *** p<0.001. (B) Representative images from liver section stained for nuclei using DAPI and for FIX using Rhodamine. (C) Upper image: Liver of HB mice treated with saline buffer as control for the specificity of the FIX staining. Lower image: Liver section from the 5 minutes timepoint and rFIXK5A treated group. The portal triad is circumscribed by a dotted line: 1-branch of hepatic portal vein, 2-branch of hepatic artery, and branch of bile duct. FIX positive regions are indicated by arrows.

FIG. 4: Pharmacokinetic profile of fusion FIX variant polypeptides in HB mice after intravenous administration. HB mice (n=3) were injected with rFIX antigen dose of 200 IU/kg of rFIXWT-FP, rFIXK5A-FP and rFIXK5R-FP via the lateral tail vein. Samples were collected at different timepoints until 168 hours post administration. Timepoints up to 168 hours of blood sampling are plotted in the X axis. LLOQ refer to the lowest concentration of rFIX:Ag that can be reliably detected based on ELISA. Each point represents the mean±SD of n=1-3 mice.

FIG. 5: Pharmacokinetic profile of fusion FIX variant polypeptides in HB mice after subcutaneous administration. HB mice (n=3) were injected with rFIX antigen dose of 200 IU/kg of rFIXWT-FP, rFIXK5A-FP and rFIXK5R-FP in the neck. Samples were collected at different timepoints until 168 hours post administration. Timepoints up to 168 hours of blood sampling are plotted in the X axis. LLOQ refer to the lowest concentration of rFIX:Ag that can be reliably detected based on ELISA. Each data point represents the mean±SD of n=1-3 mice. Only 1 out of 3 animals (a) had detectable levels in the rFIXWT and rFIXK5A group at 168 hours.

FIG. 6: Comparison of the hemostatic efficacy in a tail clip bleeding model after subcutaneous administration of a vehicle control, rFIXWT, rFIXK5A, and rFIXK5R. The group size was n=9-10 animals and for statistical analysis the treatment groups were compared to the vehicle group. (A) The blood loss normalized to the body weight (gram) is depicted in a scatter plot. Each bar represents the median. (B) The bleeding incidence, determined by time to stop bleeding over 30 minutes observation period is plotted using a Kaplan-Meier curve and statistical analysis was performed using a Log-rank (Mantel-Cox) test. (C) The (adjusted) P values were summarized in the tables below the respective graphs (in FIG. 6B) at each timepoint and were highlighted in bold if significant (p<0.05). For blood loss, statistical analysis was performed using a 1-way ANOVA-test followed by a Dunnett's post hoc test. BI is the abbreviation for bleeding incidence and the statistical analysis for BI was performed using a Log-rank (Mantel-Cox) test. Vehicle at timepoints 24 hour and 168 hours were extracted from historical data (under the similar conditions).

FIG. 7: Comparison of the hemostatic efficacy at different timepoints (0.25-336 hours post administration) after intravenous administration of hemophilia B mice with of a vehicle control, rFIXWT, rFIXK5A, and rFIXK5R in a tail clip bleeding model. The group size was n=8-10 animals and for statistical analysis the treatment groups were compared to the vehicle group. (A) The blood loss normalized to the body weight (gram) is depicted in a scatter plot. Each point represents the median. (B) The efficacy of rFIX to stop bleeding over 30 minutes is plotted using a Kaplan-Meier curve. (C) The (adjusted) P values were summarized in the tables below the respective graphs at each timepoint and were highlighted in bold if significant (p<0.05). Statistics were performed using a 1-way ANOVA-test followed by a Dunnett's post hoc test for the blood loss parameter. BI is the abbreviation for bleeding incidence and the statistical analysis was performed using a Log-rank (Mantel-Cox).

FIG. 8: Exposure of rFIX in plasma of HB mice after tail clip model (intravenous administration). HB mice were administered intravenously with rFIXWT, rFIXK5A, and rFIXK5R. At the end of each experiment blood was collected from the injured animals for measurements of antigen levels (FIX:Ag, solid lines), and activity levels (FIX:C, dashed lines). The antigen levels in the rFIXWT group at 24 hours is not available (+) because no blood samples were collected. Each symbol represents the median±95% CI of 8-10 animals. The LLOQ of FIX:Ag (6.25 mlU/mL) and of the FIX:C (100 mlU/mL) is indicated with black dotted lines against left and right Y-axis, respectively. No FIX:Ag levels were detectable in plasma at 336 hours, LLOQ differed at this timepoint and for all three proteins was 25 mlU/mL (*). Values ≤LLOQ were plotted as 100 mlU/mL for FIX:C or 6.25 mlU/mL for FIX:Ag.

### EXAMPLES

The following examples are provided to illustrate various embodiments of the present invention. The examples are illustrative and are not intended to limit the invention in any way.

### Example 1—Subcutaneous and Intravenous Administration of Wild-Type FIX and FIX Variant Polypeptides in a Mouse Model of Hemophilia

A murine model of hemophilia (ref 55) was used to analyse the pharmacokinetic profile of FIX variant polypeptides when administered either subcutaneously or intravenously. This model is referred to herein as “HB mice”.

Recombinant FIX variant polypeptides modified at position 5 of the Gla domain with alanine (rFIXK5A) or arginine (rFIXK5R) were tested. The wild type FIX polypeptide used was a marketed rFIXWT product (BeneFIX®). HB mice were injected with a dose of 25 nmol/kg rFIXWT, rFIXK5A and rFIXK5R intravenously via the lateral tail vein or subcutaneously in the neck. Blood samples were collected at several timepoints: 5 minutes, 2 hr, 6 hr, 24 hr, 48 hr, 72 hr, 120 hr, and 144 hr (6 days), 168 hr (7 days), 240 hr (10 days), and 336 hr (14 days). Blood samples to generate plasma were taken retro-orbitally at all timepoints, and additionally terminally by puncturing of the vena cava under deep anaesthesia (Ketamin 65 mg/kg, Xylazin 13 mg/kg, and Acepromazin 2 mg/kg, mixed in the same syringe and given i.p.).

The pharmacokinetic profiles of the FIX polypeptides were determined by measuring rFIX antigen levels at various timepoints.

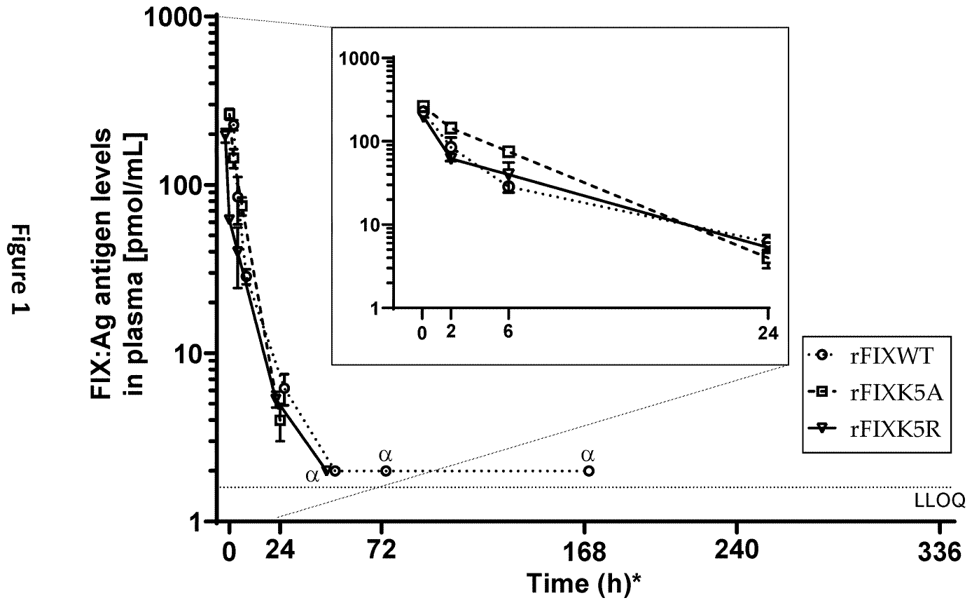

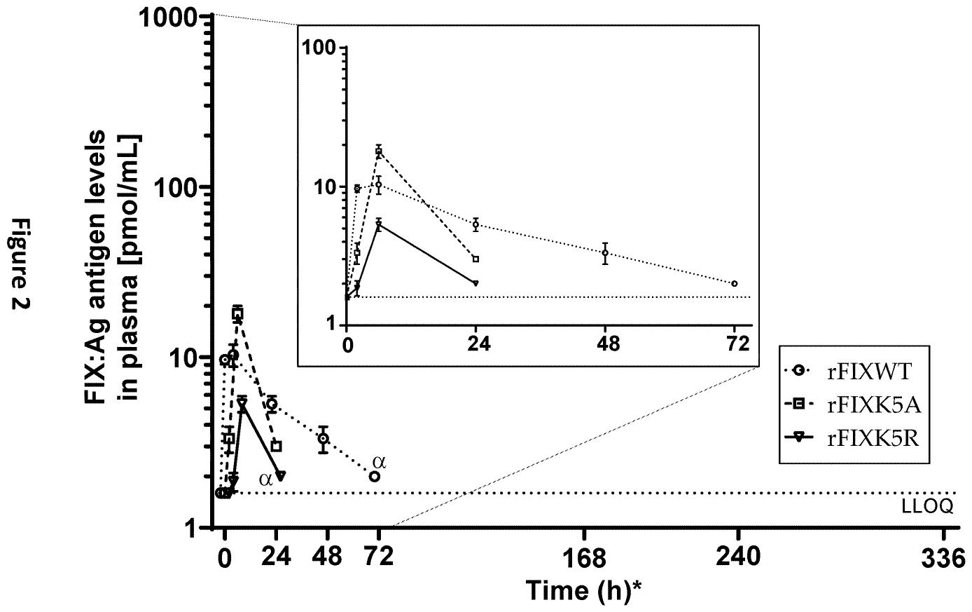

As shown in FIG. 1, in general, the plasma exposure of rFIX when administered intravenously was comparable for all proteins (rFIXWT, rFIXK5A and rFIXK5R). However, when focusing on the early timepoints (insert in FIG. 1) it is evident that the clearance of rFIXK5A was monophasic whereas rFIXWT and rFIXK5R had a biphasic profile. The biphasic profile suggested that rFIXWT and rFIXK5R were being distributed at a faster rate to the extravascular compartment during the initial phase.

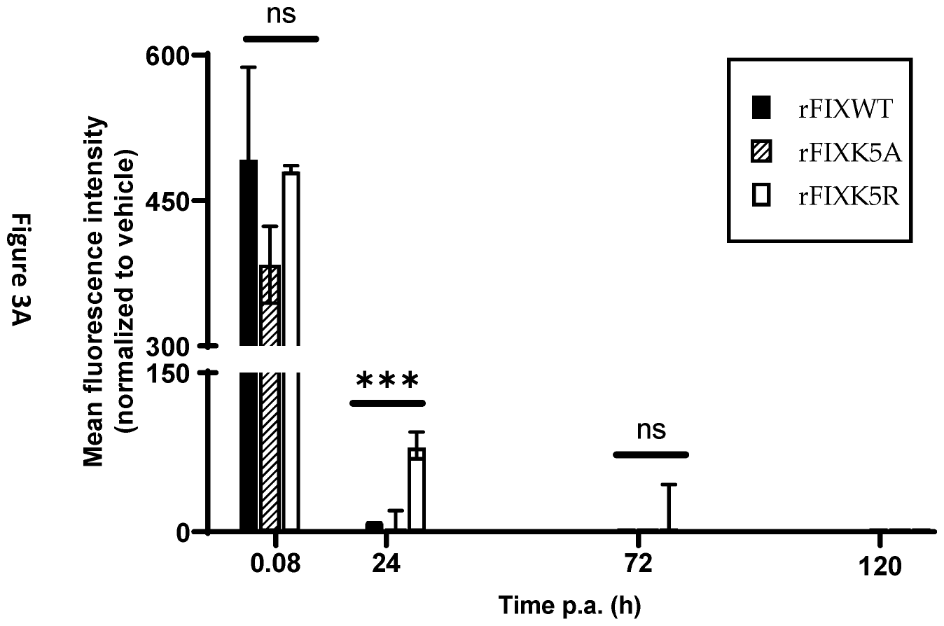

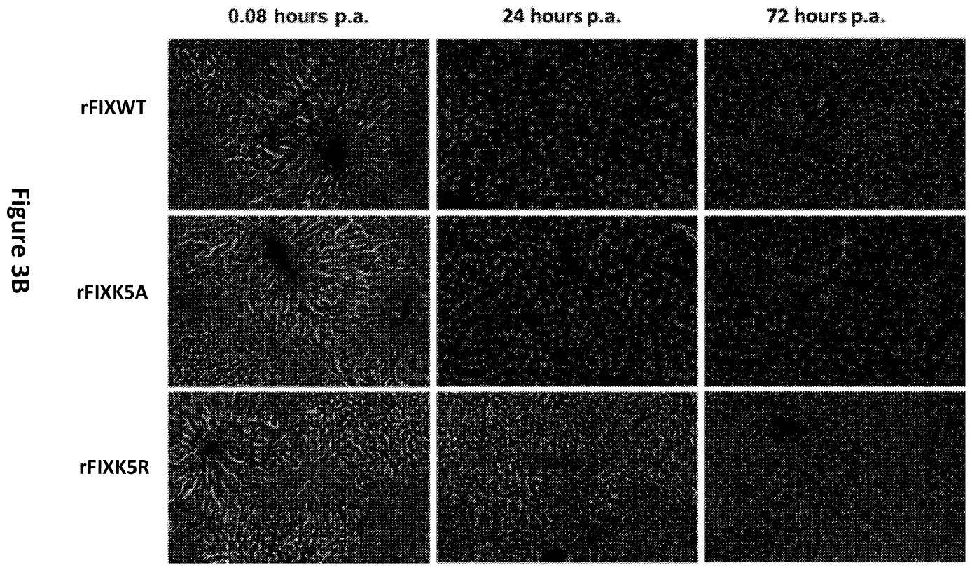

Interestingly, the K5A mutation had a positive effect on plasma exposure when administered subcutaneously, compared to rFIXWT and rFIXK5R (FIG. 2). The area under the plasma drug concentration-time curve (which reflects the actual plasma exposure of the FIX proteins) is clearly higher for rFIXK5A than rFIXK5R. All together these results suggest that after subcutaneous administration rFIXK5A enters the circulation more easily, whereas the release of rFIXK5R to the circulation from the subcutaneous site of injection is slower and less efficient. Without wishing to be bound by any particular theory, the reason that rFIXK5A enters the circulation more easily appears to be because that variant binds extracellular components to a weaker extent.

### Non-Compartmental PK Analysis

Plasma levels of FIX antigen after intravenous administration were used for non-compartmental PK analysis as summarized in Table 2.

TABLE 2
Non-compartmental analysis of intravenously
administered FIX polypeptides

| PK parameter | Unit | rFIXWT | rFIXK5A | rFIXK5R |
|---|---|---|---|---|
| AUC_0-last | h*pmol/mL | 981 ± 112 | 1560 ± 100 | 955 ± 21 |
| AUC_inf | h*pmol/mL | 995 ± 111 | 1590 ± 103 | 966 ± 22 |
| extrap | % | 1.4 ± 2 | 1.6 ± 0.3 | 1.2 ± 0.8 |
| Cmax_pred | pmol/mL | 233 ± 51 | 268 ± 17 | 203 ± 16 |
| Clearance | mL/h/kg | 25.1 ± 3.0 | 15.8 ± 1.0 | 25.9 ± 0.8 |
| t½_term | h | 13.8 ± 8 | 4.24 ± 0.2 | 8.02 ± 0.3 |
| MRT | h | 9.55 ± 4 | 4.68 ± 0.2 | 7.34 ± 0.4 |
| IVR | % | 37.2 ± 8 | 42.9 ± 3 | 32.4 ± 3 |
| Vc | mL/kg | 107 ± 63 | 93.6 ± 6 | 123 ± 9 |
| Vss | mL/kg | 240 ± 110 | 73.9 ± 4 | 190 ± 13 |
| Vz | mL/kg | 499 ± 285 | 96.8 ± 7 | 299 ± 15 |

The total exposure after intravenous injection was evidently higher for rFIXK5A which exhibited an area under the curve (AUC), from the time 0 to the last measurable concentration (AUC_0-last), of ˜1560 h*pmol/mL, followed by lower AUC levels of rFIXWT (˜981 h*pmol/mL) and rFIXK5R (˜955 h*pmol/mL). The predicted maximum concentration (Cmax_pred) of rFIX was measured to be highest for the rFIXK5A (˜268 pmol/mL) group, succeeded by rFIXWT (˜233 pmol/mL) and rFIXK5R (˜203 pmol/mL).

In general, rFIXK5A reached ca. 32% higher Cmax_pred compared to rFIXK5R and the total exposure over time was ca. 63% and 65% higher for rFIXK5A compared to rFIXK5R (AUC_0-last and AUC_0-inf; Table 2). rFIXK5A exhibited the shortest terminal half-life (˜4 hours), the lowest systemic clearance (˜16 mL/h/mg), and the lowest mean retention time (MRT) (˜4.68 hours) compared to rFIXWT and rFIXK5R (Table 2). However, rFIXK5A had the highest in vivo recovery (˜42.9%), while rFIXWT (37.2%) and rFIXK5A (32.4%) exhibited a slightly lower in vivo recovery (IVR) as shown in Table 2.

The apparent volume of distribution in the central compartment (Vc), at steady state (Vss), and during the terminal elimination phase (Vz) were lowest for rFIXK5A, which suggests low tissue distribution of rFIXK5A. The fraction of dose absorbed by the extravascular compartment can only be estimated (indirectly) based on the plasma levels measurements.

These results again suggest that lower levels of rFIXK5A bind to the extravascular compartment compared to rFIXWT and rFIXK5R.

### Example 2—Tissue Absorption of FIX Variant Polypeptides

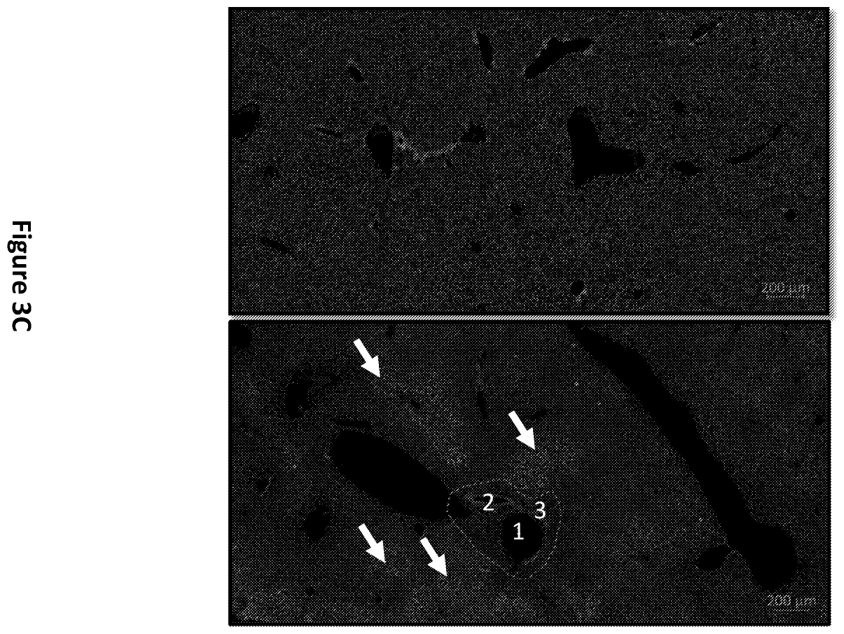

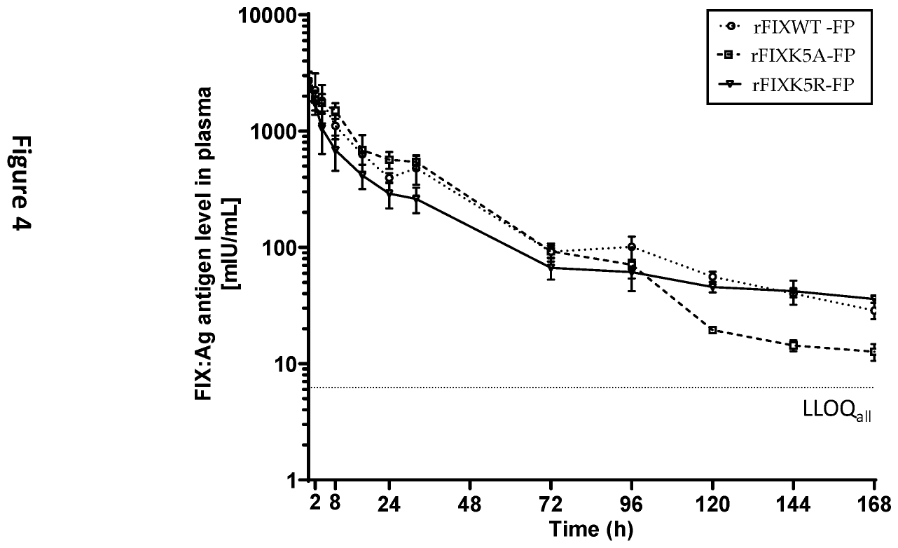

Samples from the livers of the HB mice that were intravenously and subcutaneously treated in Example 1 were taken at 0.08 hr, 24 hours and 72 hours post administration as shown in FIG. 3A. Samples were processed and quantification of FIX immunostaining was performed in at least 6 independent sections. Representative images of these samples are provided in FIG. 3B.

The HB mice which were subcutaneously treated had a low signal-to-noise ratio, which made the quantification of the FIX immunostaining unreliable.

The specificity of the FIX staining was confirmed in liver sections from HB mice that received previously saline buffer (human FIX negative samples). Those sections served as negative control and were stained under identical conditions (including primary and secondary antibodies) as liver samples of mice treated previously with rFIX. Only samples from animals treated previously with human rFIX displayed a signal (FIG. 3C).

In the intravenous group, 15 minutes following injection, all three rFIX proteins were detected in the liver with similar mean fluorescence intensities. However, at 24 hours following intravenous injection, only rFIXK5R was detected in the liver sections with a strong and robust signal, while faint signals were detected under identical conditions in the rFIXK5A and rFIXWT treated groups (FIG. 3B). This data shows that rFIXK5A and rFIXWT were cleared from the liver at a faster rate than rFIXK5R.

### Example 3—Subcutaneous and Intravenous Administration of Fusion FIX Variant Polypeptides in a Mouse Model

FIX variant polypeptides modified at position 5 of the Gla domain with alanine (rFIXK5A-FP) and arginine (rFIXK5R-FP) fused to albumin were tested and compared with rFIXWT-FP (IDELVION®).

HB mice were injected with a dose of 200 IU/kg (21 nmol/kg for rFIXWT-FP, 15 nmol/kg for rFIXK5A-FP, and 19 nmol/kg for rFIXK5R-FP) based on antigen concentration. The antigen concentration (rFIX:Ag) of each protein was previously determined using ELISA against rFIXWT-FP with a known concentration (one stage clotting potency, FIX:C). The measured concentrations (FIX:Ag) of rFIXK5A-FP (840 IU/mL), rFIXK5R-FP (722 IU/mL), and rFIXWT-FP (229 IU/mL) were diluted as needed and administered intravenously via the lateral vein or subcutaneously in the neck. Blood samples were collected from the saphenous vein at several timepoints: 5 min, 2 hr, 8 hr, 16 hr, 24 hr, 32 hr, 72 hr, 96 hr, 120 hr (5 days), 144 hr (6 days), and 168 hr (7 days). Samples taken from the saphenous vein were collected in EDTA tubes (Sarstedt Microvette CB 300 DI-Kalium-EDTA). Subcutaneous and intravenous pharmacokinetic profiles of rFIXWT-FP, rFIXK5A-FP, and rFIXK5R-FP were determined by measuring rFIX antigen levels (FIX:Ag) at various timepoints.

When administered intravenously, rFIXK5R-FP was rapidly eliminated from the plasma in the initial phase, albeit at slower rate than the unfused protein, which is likely to be due to the half-life prolongation effect of the albumin. The rFIXK5A-FP exhibited a slower elimination from the plasma and more linear initial distribution phase.

Overall, the progression of the curves is very similar for rFIXK5R-FP and rFIXWT-FP, while rFIXK5A-FP exhibits a linear clearance from blood at initial timepoints and at later timepoints (>96 hours) the plasma concentration drops faster compared to the other two proteins.

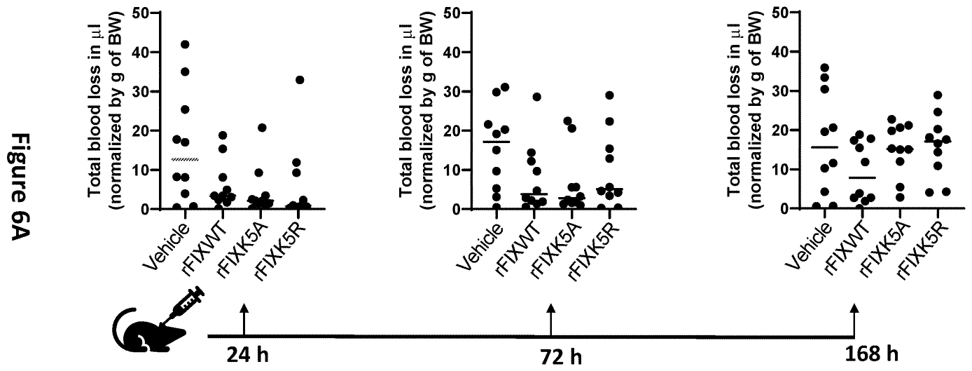

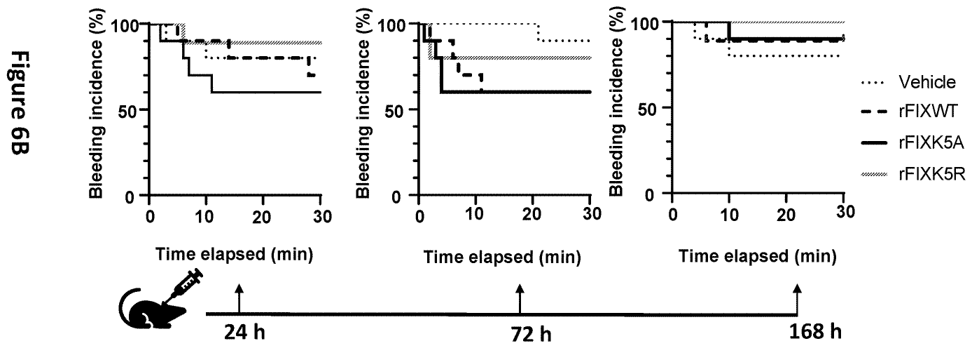

When administered subcutaneously, rFIXK5R-FP had the lowest bioavailability and exhibited the lowest Cmax and the lowest AUC (FIG. 5). These results are consistent with the observations made for the non-fused equivalent (FIG. 2). The investigation of the subcutaneous profile revealed a lower AUC_0-last (˜9 h*IU/mL), AUC_inf (˜12 h*IU/mL) and Cmax (˜0.1 IU/mL for rFIXK5R-FP compared to rFIXWT-FP (˜31 h*IU/mL; ˜31 h*IU/mL; ˜0.6 IU/mL) and rFIXK5A-FP (˜32 h*IU/mL; ˜34 h*IU/mL; ˜0.8 IU/mL).

### Non-Compartmental PK Analysis

Plasma level of rFIX (rFIX:Ag) after intravenous and subcutaneous administration was used for a non-compartmental model as summarized in Table 3 for intravenous administration and Table 4 for subcutaneous administration.

TABLE 3
Non-compartmental analysis of intravenously
administered FIX polypeptides

| PK | Unit | rFIXWT-FP | rFIXK5A-FP | rFIXK5R-FP |
|---|---|---|---|---|
| AUC_0-last | h*IU/mL | 47.6 ± 8.0 | 49.2 ± 6.2 | 31.5 ± 4.3 |
| AUC_inf | h*IU/mL | 49.3 ± 8.1 | 50.6 ± 5.1 | 37.4 ± 4.0 |
| extrap | % | 3.5 ± 0.2 | 3.0 ± 3.1 | 15.9 ± 2.6 |
| Cmax_pred | IU/mL | 2.77 ± 0.55 | 2.33 ± 0.52 | 2.72 ± 0.51 |
| Clearance | mL/h/kg | 4.1 ± 0.7 | 4.0 ± 0.4 | 5.4 ± 0.6 |
| t½_term | h | 42.7 ± 3.9 | 74.1 ± 59.6 | 114.9 ± 14.5 |
| MRT | h | 37.6 ± 3.3 | 34.5 ± 11.4 | 80.2 ± 11.8 |
| IVR | % | 54.4 ± 10.9 | 46.7 ± 10.4 | 54.4 ± 10.2 |
| Vc | mL/kg | 75.7 ± 16.8 | 88.6 ± 19.5 | 75.2 ± 13.9 |
| Vss | mL/kg | 156.4 ± 34.5 | 139.6 ± 57.4 | 436.4 ± 107.1 |
| Vz | mL/kg | 254.1 ± 44.0 | 444.4 ± 382.6 | 896.5 ± 176.1 |

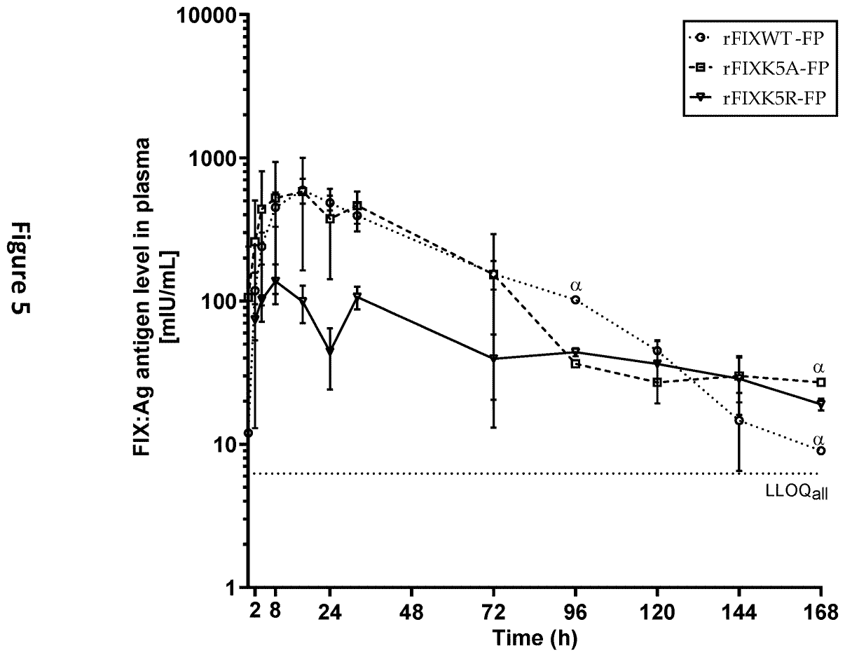

Table 3 shows that the total exposure after intravenous injection was comparable between rFIXK5A-FP and rFIXWT-FP and exhibited AUC_0-last of ˜49 h*IU/mL and ˜48 h*IU/mL, respectively, followed by the lower concentration of rFIXK5R-FP (˜32 h*IU/mL) (FIG. 4). The maximum concentration (Cmax) of rFIX was predicted to be similar for rFIXK5R-FP (˜2.7 IU/mL), rFIXWT-FP (˜2.8 IU/mL), and rFIXK5A (˜2.3 IU/mL). rFIXK5R-FP exhibited the longest half-life (˜115 h) succeeded by rFIXK5A-FP (˜74 h) and rFIXWT-FP (˜43 h). The systemic elimination (clearance) and the mean residence time (MRT) were higher for rFIXK5R-FP (˜5 mL/h/kg and ˜80 h) but comparable between rFIXK5A-FP (˜4 mL/h/kg and ˜35 h) and rFIXWT-FP (˜4 mL/h/kg and ˜38 h). rFIXK5A-FP in-vivo recovery was decreased (˜47%), while rFIXWT-FP (54%) and rFIXK5A-FP (54%) exhibited a slightly higher in-vivo recovery (IVR) as shown in Table 3.

TABLE 4
Non-compartmental analysis of subcutaneously
administered FIX polypeptides

| PK parameter | Unit | rFIXWT-FP | rFIXK5A-FP | rFIXK5R-FP |
|---|---|---|---|---|
| AUC_0-last | h*IU/mL | 30.7 ± 1.4 | 32.0 ± 7.8 | 8.9 ± 1.0 |
| AUC_inf | h*IU/mL | 31.0 ± 1.4 | 33.6 ± 8.3 | 11.5 ± 1.1 |
| extrap | % | 0.9 ± 0.1 | 4.7 ± 2.7 | 22.1 ± 15.8 |
| Cmax | IU/mL | 0.60 ± 0.12 | 0.77 ± 0.25 | 0.14 ± 0.33 |
| Clearance | mL/h/kg | 6.5 ± 0.3 | 6.2 ± 1.8 | 17.5 ± 1.6 |
| t½_term | h | 18.0 ± 3.5 | 46.6 ± 26.6 | 144.3 ± 164.1 |
| MRT | h | 42.5 ± 3.0 | 47.0 ± 6.0 | 153.2 ± 113.3 |
| BA | % | 62.9 ± 6.6 | 66.4 ± 11.5 | 30.8 ± 3.1 |

The bioavailability (BA) of the rFIXK5R-FP was lower (˜31%) than rFIXK5A-FP (66%) and rFIXWT-FP (˜63%) when subcutaneously administered (Table 4).

These results suggest that, following subcutaneous administration, rFIXK5R-FP (stronger binding in the extravascular space) potentially adheres longer at the site of injection resulting in lower plasma levels and bioavailability.

In general, the area under the curve (AUC) of the albumin-fused proteins were higher compared to the non-fused proteins, as expected. Albumin fusion prolongs the time rFIX can circulate in the body before being eliminated.

### Example 4—In Vivo Efficacy of FIX Variant Polypeptides Administered Subcutaneously and Intravenously

The hemostatic efficacy of the FIX variant polypeptides was evaluated in HB mice. The efficacy studies were conducted with non-fused rFIX proteins to enable better comparison with the published literature data on the role of extravascular FIX.

### Subcutaneous Administration

The hemostatic efficacy of rFIX proteins (rFIXWT, rFIXK5A and rFIXK5R) was evaluated at 24 hours, 72 hours, and 168 hours after subcutaneous injection of rFIX proteins (FIX:C 50 IU/kg) in a tail clip model. The plasma exposure at the end of the experiment showed no detectable rFIX antigen levels (FIX:Ag, <12.5 mlU/mL) in the samples, and only low levels of rFIX activity were detectable at 24 hours after subcutaneous administration in the clotting activity assay (FIX:C). The FIX:C activity levels were close to LLOQ and only neglectable amounts, if any, of FIX were detectable in circulation at the end of the experimental procedure.

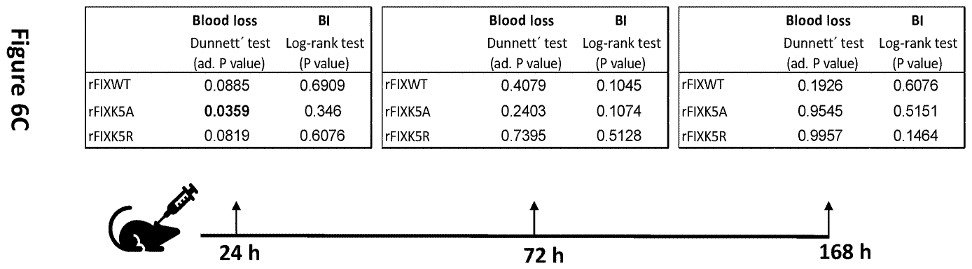

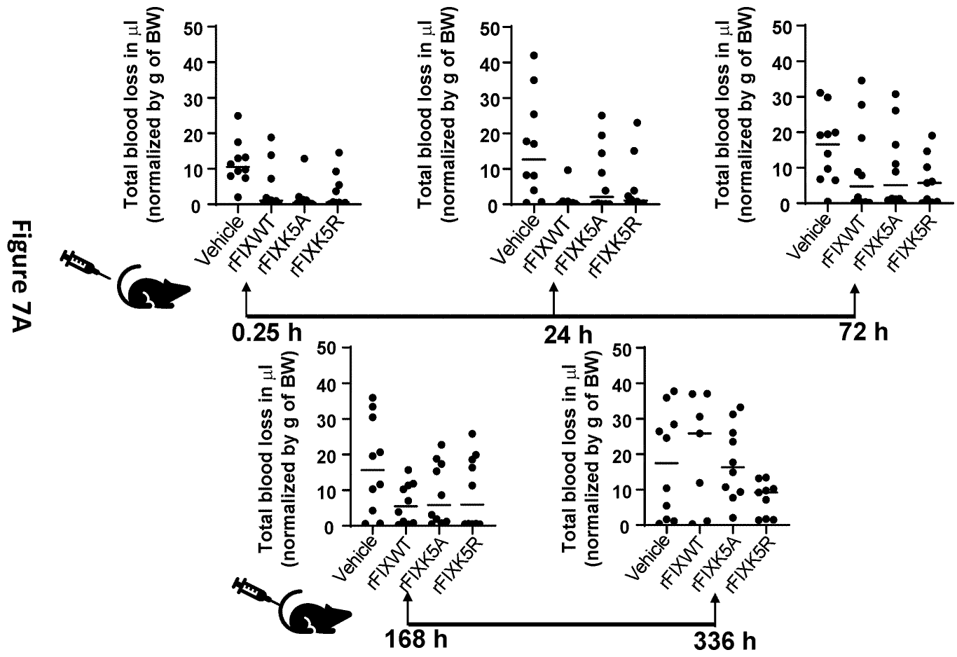

Interestingly, the subcutaneous administration of rFIXWT and rFIXK5R compromised the efficacy of these two rFIX proteins as there was no significant difference in blood loss or bleeding incidence between vehicle and rFIXWT or rFIXK5R treated groups (FIG. 6). Despite dosing at the same clotting activity, only rFIXK5A showed a statistical significance in the blood loss at 24 hours (FIG. 6C). The efficacy of rFIXK5A also diminished over time with no significant effect in blood loss after 72 hours.

The maximum reduction in bleeding incidence following subcutaneous administration was observed for rFIXWT and rFIXK5A proteins at 72 hours (Vehicle: 90%, rFIXWT: 60%, rFIXK5A: 60%, rFIXK5R: 80%, FIG. 6B and FIG. 6C). Overall, only rFIXK5A demonstrated a statistically significant efficacy after subcutaneous administration. The rFIXWT and rFIXK5R efficacy, despite providing hemostatic protection, was inferior compared to rFIXK5A.

### Intravenous Administration

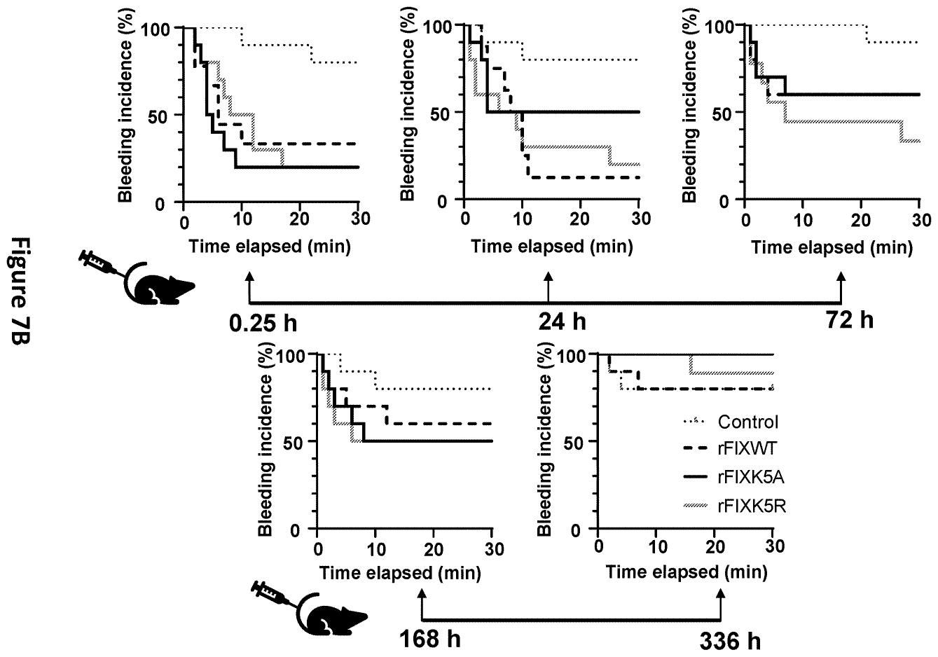

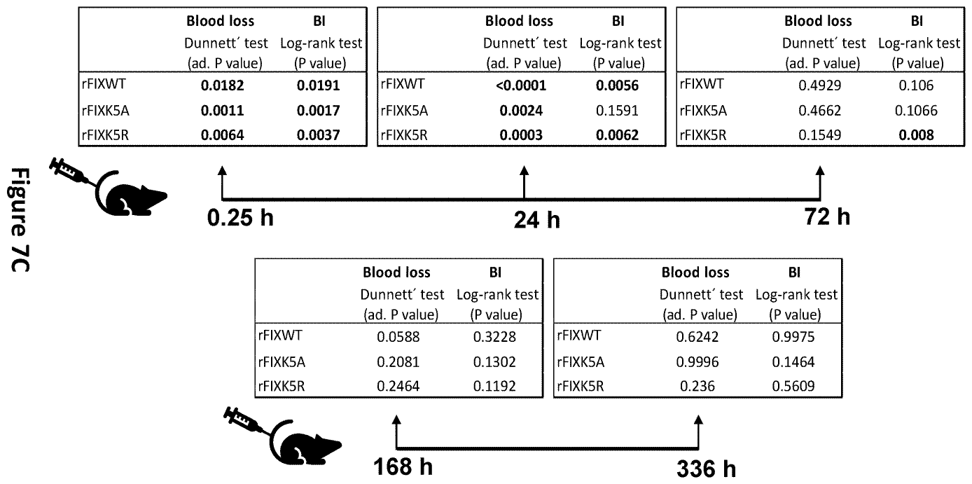

Hemostatic efficacy of rFIX proteins was evaluated at 15 minutes, 24 hours, 72 hours, 168 hours and 336 hours after i.v. injection by monitoring the total blood loss and bleeding incidence. The results of blood loss and bleeding incidence are shown in FIG. 7A and FIG. 7B, respectively. For statistical analysis, the set of rFIX molecules (rFIXWT, rFIXK5A and rFIXK5R) were compared at every timepoint individually and p values are summarized in FIG. 7C. Treatment of HB mice with rFIX significantly protected the animals from total blood loss (vehicle: ˜10 μL/g of BW; rFIX treated: ˜1 μL/g of BW, FIG. 7A) until 24 hours post treatment. As depicted in FIG. 7A, the total blood loss is comparable between rFIX treated groups until 168 hours. However, at 336 hours post-administration only the animals treated with rFIXK5R had nearly 50% decrease in blood loss compared to vehicle group, though not statistically significant (vehicle: ˜18 μL/g of BW; rFIXK5R: ˜9 μL/g of BW).

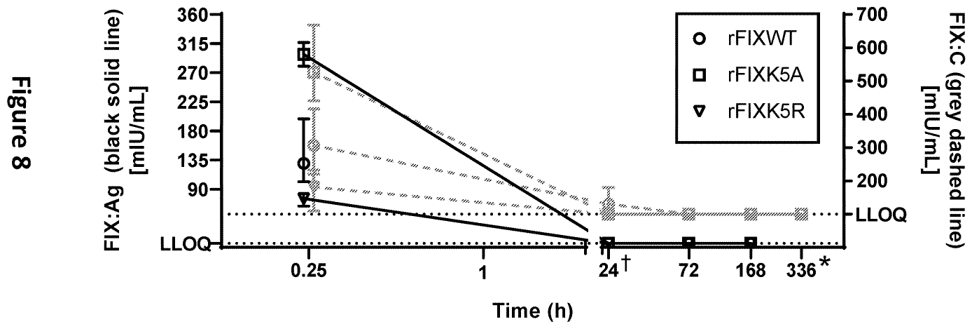

A significant effect in protection from bleeding incidence was observed for all the rFIX molecules at 15 minutes post treatment. However, at later timepoints the efficacy of individual rFIX proteins decreased over time at different rates. More specifically, the statistically significant effect on reducing bleeding incidence was lost at 24 hours post treatment for rFIXK5A, at 72 hours for rFIXWT and at 168 hours for rFIXK5R (FIG. 7C). It is worth noting that both parameters, blood loss and bleeding incidence, need to be contextualized because these parameters reflect different aspects of hemostasis. For example, the bleeding incidence (FIG. 7B) or bleeding time which indicates the time when the mice stop bleeding reflects full occlusion of the vessel. On the other hand, non-occlusive thrombus could potentially reduce blood loss (FIG. 7A), but animals may continue to bleed throughout the observation time. rFIX antigen and activity was detectable in the plasma in the 15 minutes group and no detectable rFIX protein was present in the circulation after 24 hours (FIG. 8). Hence, hemostatic efficacy observed at 24 hours and beyond is in part attributed potentially to extravascular FIX. The 2-fold difference between the FIX:Ag and FIX:C values in mlU/mL can be explained by the differences in the assays. The calibration curve of one stage clot assay (OSCA; FIX:C) is based on standard human plasma (SHP) and in ELISA the injection solution is used as a standard. Additionally, a matrix effect (normal ranges of other plasma proteins and activation of plasma proteins involved in the intrinsic pathway of the coagulation cascade) using OSCA cannot be excluded due to the conditions under which mouse plasma samples were collected (after transection of a main peripheral artery and veins leading to major bleeding and consumption of coagulation factors).

In general, all proteins were comparable regarding their ability to reduce blood loss following intravenous administration. Though statistically not significant, rFIXK5R showed the longest efficacy in reducing blood loss until 14 days compared to vehicle group, even when no rFIX was detected in the circulation. This result suggests the presence of non-circulating but accessible rFIX to the site of injury. Beyond blood loss, the bleeding incidence suggest that rFIXK5A conveys good protection but loses this ability very quickly, demonstrating that rFIXK5A may not be easily accessible at the site of injury after 24 hours following intravenous administration, therefore, limiting the growth of a stable clot that requires both circulating FIX in the thrombotic area and easily accessible FIX at the site, to achieve full vessel occlusion.

It will be understood that the invention has been described by way of example only and modifications may be made whilst remaining within the scope and spirit of the invention.

SEQUENCES
Human wild-type FIX polypeptide
SEQ ID NO: 1
YNSGKLEEFVQGNLERECMEEKCSFEEAREVFENTERTTEFWKQYVDGDQ
CESNPCLNGGSCKDDINSYECWCPFGFEGKNCELDVTCNIKNGRCEQFCK
NSADNKVVCSCTEGYRLAENQKSCEPAVPFPCGRVSVSQTSKLTRAETVF
PDVDYVNSTEAETILDNITQSTQSENDFTRVVGGEDAKPGQFPWQVVLNG
KVDAFCGGSIVNEKWIVTAAHCVETGVKITVVAGEHNIEETEHTEQKRNV
IRIIPHHNYNAAINKYNHDIALLELDEPLVLNSYVTPICIADKEYTNIFL
KFGSGYVSGWGRVFHKGRSALVLQYLRVPLVDRATCLRSTKFTIYNNMFC
AGFHEGGRDSCQGDSGGPHVTEVEGTSFLTGIISWGEECAMKGKYGIYTK
VSRYVNWIKEKTKLT
Coding sequence for human wild-type FIX
polypeptide
SEQ ID NO: 2
ATGTATAATTCAGGTAAATTGGAAGAGTTTGTTCAAGGGAACCTTGAGAG
AGAATGTATGGAAGAAAAGTGTAGTTTTGAAGAAGCACGAGAAGTTTTTG
AAAACACTGAAAGAACAACTGAATTTTGGAAGCAGTATGTTGATGGAGAT
CAGTGTGAGTCCAATCCATGTTTAAATGGCGGCAGTTGCAAGGATGACAT
TAATTCCTATGAATGTTGGTGTCCCTTTGGATTTGAAGGAAAGAACTGTG
AATTAGATGTAACATGTAACATTAAGAATGGCAGATGCGAGCAGTTTTGT
AAAAATAGTGCTGATAACAAGGTGGTTTGCTCCTGTACTGAGGGATATCG
ACTTGCAGAAAACCAGAAGTCCTGTGAACCAGCAGTGCCATTTCCATGTG
GAAGAGTTTCTGTTTCACAAACTTCTAAGCTCACCCGTGCTGAGACTGTT
TTTCCTGATGTGGACTATGTAAATTCTACTGAAGCTGAAACCATTTTGGA
TAACATCACTCAAAGCACCCAATCATTTAATGACTTCACTCGGGTTGTTG
GTGGAGAAGATGCCAAACCAGGTCAATTCCCTTGGCAGGTTGTTTTGAAT
GGTAAAGTTGATGCATTCTGTGGAGGCTCTATCGTTAATGAAAAATGGAT
TGTAACTGCTGCCCACTGTGTTGAAACTGGTGTTAAAATTACAGTTGTCG
CAGGTGAACATAATATTGAGGAGACAGAACATACAGAGCAAAAGCGAAAT
GTGATTCGAATTATTCCTCACCACAACTACAATGCAGCTATTAATAAGTA
CAACCATGACATTGCCCTTCTGGAACTGGACGAACCCTTAGTGCTAAACA
GCTACGTTACACCTATTTGCATTGCTGACAAGGAATACACGAACATCTTC
CTCAAATTTGGATCTGGCTATGTAAGTGGCTGGGGAAGAGTCTTCCACAA
AGGGAGATCAGCTTTAGTTCTTCAGTACCTTAGAGTTCCACTTGTTGACC
GAGCCACATGTCTTCGATCTACAAAGTTCACCATCTATAACAACATGTTC
TGTGCTGGCTTCCATGAAGGAGGTAGAGATTCATGTCAAGGAGATAGTGG
GGGACCCCATGTTACTGAAGTGGAAGGGACCAGTTTCTTAACTGGAATTA
TTAGCTGGGGTGAAGAGTGTGCAATGAAAGGCAAATATGGAATATATACC
AAGGTATCCCGGTATGTCAACTGGATTAAGGAAAAAACAAAGCTCACTTA
A
Human wild-type Factor IX that includes the
signal and the propeptide
SEQ ID NO: 3
MQRVNMIMAESPGLITICLLGYLLSAECTVELDHENANKILNRPKRYNSG
KLEEFVQGNLERECMEEKCSFEEAREVFENTERTTEFWKQYVDGDQCESN
PCLNGGSCKDDINSYECWCPFGFEGKNCELDVTCNIKNGRCEQFCKNSAD
NKVVCSCTEGYRLAENQKSCEPAVPFPCGRVSVSQTSKLTRAETVFPDVD
YVNSTEAETILDNITQSTQSENDETRVVGGEDAKPGQFPWQVVLNGKVDA
FCGGSIVNEKWIVTAAHCVETGVKITVVAGEHNIEETEHTEQKRNVIRII
PHHNYNAAINKYNHDIALLELDEPLVLNSYVTPICIADKEYTNIFLKFGS
GYVSGWGRVFHKGRSALVLQYLRVPLVDRATCLRSTKFTIYNNMFCAGFH
EGGRDSCQGDSGGPHVTEVEGTSFLTGIISWGEECAMKGKYGIYTKVSRY
VNWIKEKTKLT
Coding sequence for human wild-type Factor
IX that includes the signal and
the propeptide
SEQ ID NO: 4
ATGCAGCGCGTGAACATGATCATGGCAGAATCACCAGGCCTCATCACCAT
CTGCCTTTTAGGATATCTACTCAGTGCTGAATGTACAGTTTTTCTTGATC
ATGAAAACGCCAACAAAATTCTGAATCGGCCAAAGAGGTATAATTCAGGT
AAATTGGAAGAGTTTGTTCAAGGGAACCTTGAGAGAGAATGTATGGAAGA
AAAGTGTAGTTTTGAAGAAGCACGAGAAGTTTTTGAAAACACTGAAAGAA
CAACTGAATTTTGGAAGCAGTATGTTGATGGAGATCAGTGTGAGTCCAAT
CCATGTTTAAATGGCGGCAGTTGCAAGGATGACATTAATTCCTATGAATG
TTGGTGTCCCTTTGGATTTGAAGGAAAGAACTGTGAATTAGATGTAACAT
GTAACATTAAGAATGGCAGATGCGAGCAGTTTTGTAAAAATAGTGCTGAT
AACAAGGTGGTTTGCTCCTGTACTGAGGGATATCGACTTGCAGAAAACCA
GAAGTCCTGTGAACCAGCAGTGCCATTTCCATGTGGAAGAGTTTCTGTTT
CACAAACTTCTAAGCTCACCCGTGCTGAGACTGTTTTTCCTGATGTGGAC
TATGTAAATTCTACTGAAGCTGAAACCATTTTGGATAACATCACTCAAAG
CACCCAATCATTTAATGACTTCACTCGGGTTGTTGGTGGAGAAGATGCCA
AACCAGGTCAATTCCCTTGGCAGGTTGTTTTGAATGGTAAAGTTGATGCA
TTCTGTGGAGGCTCTATCGTTAATGAAAAATGGATTGTAACTGCTGCCCA
CTGTGTTGAAACTGGTGTTAAAATTACAGTTGTCGCAGGTGAACATAATA
TTGAGGAGACAGAACATACAGAGCAAAAGCGAAATGTGATTCGAATTATT
CCTCACCACAACTACAATGCAGCTATTAATAAGTACAACCATGACATTGC
CCTTCTGGAACTGGACGAACCCTTAGTGCTAAACAGCTACGTTACACCTA
TTTGCATTGCTGACAAGGAATACACGAACATCTTCCTCAAATTTGGATCT
GGCTATGTAAGTGGCTGGGGAAGAGTCTTCCACAAAGGGAGATCAGCTTT
AGTTCTTCAGTACCTTAGAGTTCCACTTGTTGACCGAGCCACATGTCTTC
GATCTACAAAGTTCACCATCTATAACAACATGTTCTGTGCTGGCTTCCAT
GAAGGAGGTAGAGATTCATGTCAAGGAGATAGTGGGGGACCCCATGTTAC
TGAAGTGGAAGGGACCAGTTTCTTAACTGGAATTATTAGCTGGGGTGAAG
AGTGTGCAATGAAAGGCAAATATGGAATATATACCAAGGTATCCCGGTAT
GTCAACTGGATTAAGGAAAAAACAAAGCTCACTTAA
Human Albumin
SEQ ID NO: 5
DAHKSEVAHRFKDLGEENFKALVLIAFAQYLQQCPFEDHVKLVNEVTEFA
KTCVADESAENCDKSLHTLFGDKLCTVATLRETYGEMADCCAKQEPERNE
CFLQHKDDNPNLPRLVRPEVDVMCTAFHDNEETFLKKYLYEIARRHPYFY
APELLFFAKRYKAAFTECCQAADKAACLLPKLDELRDEGKASSAKQRLKC
ASLQKFGERAFKAWAVARLSQRFPKAEFAEVSKLVTDLTKVHTECCHGDL
LECADDRADLAKYICENQDSISSKLKECCEKPLLEKSHCIAEVENDEMPA
DLPSLAADFVESKDVCKNYAEAKDVFLGMFLYEYARRHPDYSVVLLLRLA
KTYETTLEKCCAAADPHECYAKVFDEFKPLVEEPQNLIKQNCELFEQLGE
YKFQNALLVRYTKKVPQVSTPTLVEVSRNLGKVGSKCCKHPEAKRMPCAE
DYLSVVLNQLCVLHEKTPVSDRVTKCCTESLVNRRPCFSALEVDETYVPK
EFNAETFTFHADICTLSEKERQIKKQTALVELVKHKPKATKEQLKAVMDD
FAAFVEKCCKADDKETCFAEEGKKLVAASQAALGL
Fc polypeptide
SEQ ID NO: 6
EPKSCDKTHTCPPCPAPELLGGPSVFLFPPKPKDTLMISRTPEVTCVVVD
VSHEDPEVKENWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG
Fc polypeptide
SEQ ID NO: 7
DKTHTCPPCPAPELLGGPSVFLFPPKPKDTLMISRTPEVTCVVVDVSHED
PEVKENWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPG
Cleavable linker
SEQ ID NO: 8
PVSQTSKLTRAETVFP
K5A human FIX variant polypeptide
SEQ ID NO: 9
YNSGALEEFVQGNLERECMEEKCSFEEAREVFENTERTTEFWKQYVDGDQ
CESNPCLNGGSCKDDINSYECWCPFGFEGKNCELDVTCNIKNGRCEQFCK
NSADNKVVCSCTEGYRLAENQKSCEPAVPFPCGRVSVSQTSKLTRAETVF
PDVDYVNSTEAETILDNITQSTQSENDFTRVVGGEDAKPGQFPWQVVLNG
KVDAFCGGSIVNEKWIVTAAHCVETGVKITVVAGEHNIEETEHTEQKRNV
IRIIPHHNYNAAINKYNHDIALLELDEPLVLNSYVTPICIADKEYTNIFL
KFGSGYVSGWGRVFHKGRSALVLQYLRVPLVDRATCLRSTKFTIYNNMFC
AGFHEGGRDSCQGDSGGPHVTEVEGTSFLTGIISWGEECAMKGKYGIYTK
VSRYVNWIKEKTKLT
R338L human FIX variant polypeptide
SEQ ID NO: 10
YNSGKLEEFVQGNLERECMEEKCSFEEAREVFENTERTTEFWKQYVDGDQ
CESNPCLNGGSCKDDINSYECWCPFGFEGKNCELDVTCNIKNGRCEQFCK
NSADNKVVCSCTEGYRLAENQKSCEPAVPFPCGRVSVSQTSKLTRAETVF
PDVDYVNSTEAETILDNITQSTQSENDFTRVVGGEDAKPGQFPWQVVLNG
KVDAFCGGSIVNEKWIVTAAHCVETGVKITVVAGEHNIEETEHTEQKRNV
IRIIPHHNYNAAINKYNHDIALLELDEPLVLNSYVTPICIADKEYTNIFL
KFGSGYVSGWGRVFHKGRSALVLQYLRVPLVDRATCLLSTKFTIYNNMFC
AGFHEGGRDSCQGDSGGPHVTEVEGTSFLTGIISWGEECAMKGKYGIYTK
VSRYVNWIKEKTKLT
A CTP sequence
SEQ ID NO: 11
SSSSKAPPPS
A CTP sequence
SEQ ID NO: 12
DPRFQDSSSSKAPPPSLPSPSRLPGPSDTPIL
A CTP sequence
SEQ ID NO: 13
SSSSKAPPPSLPSPSRLPGPSDTPILPQ
A XTEN72 sequence
SEQ ID NO: 14
GAPTSESATPESGPGSEPATSGSETPGTSESATPESGPGSEPATSGSETP
GTSESATPESGPGTSTEPSEGSAPGASS
A XTEN sequence
SEQ ID NO: 15
GAPGSPAGSPTSTEEGTSESATPESGPGSEPATSGSETPASS
Non-cleavable linker
SEQ ID NO: 16
GGGGGGV
Light chain human activated Factor IX
SEQ ID NO: 17
YNSGKLEEFVQGNLERECMEEKCSFEEAREVFENTERTTEFWKQYVDGDQ
CESNPCLNGGSCKDDINSYECWCPFGFEGKNCELDVTCNIKNGRCEQFCK
NSADNKVVCSCTEGYRLAENQKSCEPAVPFPCGRVSVSQTSKLTR
Heavy chain human activated Factor IX
SEQ ID NO: 18
VVGGEDAKPGQFPWQVVLNGKVDAFCGGSIVNEKWIVTAAHCVETGVKIT
VVAGEHNIEETEHTEQKRNVIRIIPHHNYNAAINKYNHDIALLELDEPLV
LNSYVTPICIADKEYTNIFLKFGSGYVSGWGRVFHKGRSALVLQYLRVPL
VDRATCLRSTKFTIYNNMFCAGFHEGGRDSCQGDSGGPHVTEVEGTSFLT
GIISWGEECAMKGKYGIYTKVSRYVNWIKEKTKLT
R318Y/R338E/T343R (Dalcinonacog) human
FIX variant polypeptide
SEQ ID NO: 19
YNSGKLEEFVQGNLERECMEEKCSFEEAREVFENTERTTEFWKQYVDGDQ
CESNPCLNGGSCKDDINSYECWCPFGFEGKNCELDVTCNIKNGRCEQFCK
NSADNKVVCSCTEGYRLAENQKSCEPAVPFPCGRVSVSQTSKLTRAETVF
PDVDYVNSTEAETILDNITQSTQSENDFTRVVGGEDAKPGQFPWQVVLNG
KVDAFCGGSIVNEKWIVTAAHCVETGVKITVVAGEHNIEETEHTEQKRNV
IRIIPHHNYNAAINKYNHDIALLELDEPLVLNSYVTPICIADKEYTNIFL
KFGSGYVSGWGRVFHKGYSALVLQYLRVPLVDRATCLESTKFRIYNNMFC
AGFHEGGRDSCQGDSGGPHVTEVEGTSFLTGIISWGEECAMKGKYGIYTK
VSRYVNWIKEKTKLT
T148A human FIX polymorphic variant
SEQ ID NO: 20
YNSGKLEEFVQGNLERECMEEKCSFEEAREVFENTERTTEFWKQYVDGDQ
CESNPCLNGGSCKDDINSYECWCPFGFEGKNCELDVTCNIKNGRCEQFCK
NSADNKVVCSCTEGYRLAENQKSCEPAVPFPCGRVSVSQTSKLTRAEAVF
PDVDYVNSTEAETILDNITQSTQSENDFTRVVGGEDAKPGQFPWQVVLNG
KVDAFCGGSIVNEKWIVTAAHCVETGVKITVVAGEHNIEETEHTEQKRNV
IRIIPHHNYNAAINKYNHDIALLELDEPLVLNSYVTPICIADKEYTNIFL
KFGSGYVSGWGRVFHKGRSALVLQYLRVPLVDRATCLRSTKFTIYNNMFC
AGFHEGGRDSCQGDSGGPHVTEVEGTSFLTGIISWGEECAMKGKYGIYTK
VSRYVNWIKEKTKLT

### REFERENCES

[1] Schuettrumpf et al. (2005) Blood, 105, 6, 2316-2323
[2] Samelson-Jones et al. (2021) Blood 138, 3975-3975
[3] Cheung et al. (1992) J Biol Chem 267, 20529-20531
[4] Cheung et al. (1996) PNAS 93, 11068-11073
[5] Srivastava et al. (2013) Haemophilia. 2013; 19: e1-47
[6] Diagnosis of hemophilia and other bleeding disorders—a laboratory manual (World Federation of Hemophilia). In: Kitchen S, McCraw A, Echenagucia M eds. Montreal, Canada: WFH; 2010. pdf-1283.pdf (wfh.org)
[7] Stern et al. (1983) PNAS 80, 4119-4123
[8] Stern et al. (1987) Brit J Haematol 66, 227-232
[9] Chu et al. (1996), J Clin Invest 98, 1619-1625
[10] Stern et al. (1983) PNAS 80, 4119-4123
[11] Wolberg and Stafford (1997) J Biol Chem 272, 16717-16720
[12] Ahmad et al. (1989) J Biol Chem 264, 3244-3251
[13] Cooley et al. (2019) Blood 133, 2445-2451
[14] Feng et al. (2013) J Thromb Haemost 11:2176-8
[15] Gui et al. (2009) Journal of Thrombosis and Haemostasis, 7:1843-1851
[16] Mann et al. (2021) Haemophilia. 2021; 00:1-8
[17] Simioni et al. (2009) N Engl J Med. 361 (17): 1671-1675
[18] WO 2020/187969 A1
[19] Neurath and Walsh (1976) PNAS 73 (11): 3825-3832
[20] en Dunnen et al. (2016) Hum Mutat. 37 (6): 564-569
[21] Samelson-Jones and Arruda (2019) Molecular Therapy 12, 184-201
[22] Perot et al. (2015) Thromb. Res. 135, 1017-1024
[23] Quade-Lyssy et al. (2014) J. Thromb. Haemost. 12, 932-942
[24] Lin et al. (2010) J. Thromb. Haemost. 8, 1773-1783
[25] Sichler et al. (2003) J. Biol. Chem. 278, 4121-4126
[26] Milanov et al. (2012) Blood 119, 602-611
[27] Misenheimer et al. (2007) Biochemistry 46, 7886-7895
[28] S.-B. Hong et al. (2016) Am. Soc. Hematology, abstract
[29] Kao et al. (2013) Thromb. Haemost. 110, 244-256
[30] Søgaard et al. (2021) Pharmaceutics 2021, 13 (10), 1722
[31] Abramson et al (2019) Science, 363 (6427), pp. 611-615
[32] https://biograil.com/
[33] U.S. Pat. No. 8,809,271
[34] Dhalla et al (2021) Drug Deliv. Transl. Res. 1-12
[35] Beattie & Dugaiczyk (1982) Gene 20:415-422
[36] WO 2005/024044
[37] Lichenstein et al. (1994) J. Biol. Chem. 269:18149-18154
[38] Cooke & David (1985) J. Clin. Invest. 76:2420-2424
[39] WO 2005/001025
[40] Powell et al. (2013) N. Engl. J. Med., 369:2313-2323
[41] Peters et al. (2010) Blood 115:2057-2064
[42] Shapiro et al. (2012) Blood 119:666-672
[43] Fares et al. (1992) PNAS 15; 89 (10): 4304-4308
[44] Calo et al. (2015) Precision Medicine, 2, e989
[45] WO 2011/004361
[9] Schellenberger et al. (2009) Nature Biotechnology 27, 1186-1190
[47] WO 2017/024060
[48] WO 2012/006624
[49] WO 2015/106052
[50] Collins et al. (2014) Blood 124:3880-3886
[51] WO 2006/127896
[52] WO 2005/055950
[53] DeFrees et al. (2006) Glycobiology 16 (9): 833-843
[54] Gennaro (2000) Remington: The Science and Practice of Pharmacy. 20th edition, ISBN: 0683306472.
[55] Lin et al. (1997). Blood 90, 3962-3966.
[56] Bezemer et al. (2009). Haematologica 94 (5): 693-699.

1. A method of treating or preventing a bleeding disorder in a subject in need thereof, comprising administering an effective amount of Factor IX (FIX) variant polypeptide to a soft tissue of the subject, wherein the FIX variant polypeptide comprises alanine at a position corresponding to position 5 of SEQ ID NO: 1.

2. The method according to claim 1, wherein the bleeding disorder is hemophilia B.

3. The method according to claim 1, wherein the FIX variant polypeptide further comprises lysine at a position corresponding to position 10 of SEQ ID NO: 1.

4. The method according to claim 1, wherein the FIX variant polypeptide further comprises leucine at a position corresponding to position 338 of SEQ ID NO: 1.

5. The method according to claim 1, wherein the FIX variant polypeptide further comprises valine, threonine, or tryptophan at a position corresponding to position 338 of SEQ ID NO: 1, and histidine at a position corresponding to position 410 of SEQ ID NO: 1.

6. The method according to claim 1, wherein the FIX variant polypeptide further comprises tyrosine at a position corresponding to position 318 of SEQ ID NO: 1, glutamic acid at a position corresponding to position 338 of SEQ ID NO: 1, and arginine at a position corresponding to position 343 of SEQ ID NO: 1.

7. The method according to claim 1, wherein the Factor IX variant polypeptide has at least 70% sequence identity to SEQ ID NO: 1, across the full length of SEQ ID NO: 1.

8. The method according to claim 1, wherein the FIX variant polypeptide comprises a half-life enhancing portion.

9. The method according to claim 8, wherein the FIX variant polypeptide further comprises a cleavable peptide linker between the FIX variant polypeptide and the half-life enhancing portion.

10. The method according to claim 8, wherein the half-life enhancing portion is albumin.

11. The method according to claim 1, wherein the soft tissue is skin tissue or gastrointestinal tract tissue.

12. The method according to claim 11, wherein the skin tissue is subcutaneous tissue, dermal tissue, or epidermal tissue.

13. The method according to claim 11, wherein the method comprises subcutaneously administering the FIX variant polypeptide to the subject.

14. The method according to claim 11, wherein the method comprises administering the FIX variant polypeptide into the gastrointestinal tract tissue using an oral drug delivery device.

15. The method of claim 1, wherein the FIX variant polypeptide is administered in a pharmaceutical composition comprising the FIX variant polypeptide and a pharmaceutically acceptable carrier.

16. The method according to claim 8, wherein the half-life enhancing portion is an albumin variant, an albumin derivative, a polypeptide of the albumin family, a variant of a polypeptide of the albumin family, a derivative of a polypeptide of the albumin family, an immunoglobulin without an antigen binding domain, or a polyethylene glycol.


SEQUENCE LISTING (20 sequences)

SEQ ID NO: 1 - protein, 415 aa, Homo sapiens
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 2 - DNA, 1251 bp, Homo sapiens
  source 1..1251: mol_type = other DNA
atgtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaa

SEQ ID NO: 3 - protein, 461 aa, Homo sapiens
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 4 - DNA, 1386 bp, Homo sapiens
  source 1..1386: mol_type = other DNA
atgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaa

SEQ ID NO: 5 - protein, 585 aa, Homo sapiens [sequence omitted]

SEQ ID NO: 6 - protein, 231 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 16 aa, synthetic construct
PVSQTSKLTRAETVFP

SEQ ID NO: 9 - protein, 415 aa, synthetic construct
YNSGALEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 10 - protein, 415 aa, synthetic construct
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLLSTKFTIYNNMFCAGFHEGGRDSCQGDSGGPHVTEVEGTSFLTGIISWGEECAMKGKYGIYTKVSRYVNWIKEKTKLT

SEQ ID NO: 11 - protein, 10 aa, synthetic construct
SSSSKAPPPS

SEQ ID NO: 12 - protein, 32 aa, synthetic construct
DPRFQDSSSSKAPPPSLPSPSRLPGPSDTPIL

SEQ ID NO: 13 - protein, 28 aa, synthetic construct
SSSSKAPPPSLPSPSRLPGPSDTPILPQ

SEQ ID NO: 14 - protein, 78 aa, synthetic construct
GAPTSESATPESGPGSEPATSGSETPGTSESATPESGPGSEPATSGSETPGTSESATPESGPGTSTEPSEGSAPGASS

SEQ ID NO: 15 - protein, 42 aa, synthetic construct
GAPGSPAGSPTSTEEGTSESATPESGPGSEPATSGSETPASS

SEQ ID NO: 16 - protein, 7 aa, synthetic construct
GGGGGGV

SEQ ID NO: 17 - protein, 145 aa, synthetic construct
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTR

SEQ ID NO: 18 - protein, 235 aa, synthetic construct
VVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 19 - protein, 415 aa, synthetic construct
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGYSALVLQYLRVPLVDRATCLESTKFRIYNNMFCAGFHEGGRDSCQGDSGGPHVTEVEGTSFLTGIISWGEECAMKGKYGIYTKVSRYVNWIKEKTKLT

SEQ ID NO: 20 - protein, 415 aa, synthetic construct
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAEAVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT